SEQUENCE LISTING (13 sequences)

SEQ ID NO: 1 - DNA, 272 bp, Artificial
  DNA encoding artificial miRNA precursor
acagtttgcttatgtcggatccataatatatttgacaagatactttgtttttcgatagatcttgatctgacgatggaagcagtcgagtgcgtgagcaagtcatgagttgagcagggtaaagaaaagctgctaagctatggatcccataagccctaatccttgtaaagtaaaaaaggatttggttatatggattgcatatctcaggagctttaacttgccctttaatggcttttactcttcacttgctcacgcactcgctgcatcccgggtca

SEQ ID NO: 2 - DNA, 226 bp, Artificial
  DNA encoding artificial miRNA precursor
gtgtaacagaatcgttattacctctgtcaaaacgaagaggacaagatttgaggaactaaaatgcagaatctaagagttcatgtcttcctcatagagagtgcgcggtgttaaaagcttgaagaaagcacactttaaggggattgcacgacctcttagattctccctctttctctacatatcattctcttctcttcgtttgacagaggtaataacgattttcttcttc

SEQ ID NO: 3 - DNA, 222 bp, Artificial
  DNA encoding artificial miRNA precursor
aatgatgattacgatgatgagagtctctagttgtatcagagggtcttgcatggaagaatagagaatgaggttgagtttagtctgacttggccagtttttttaccaatgaatctaattaactgattctggtgttggccaagtcagactaaactctgtttccttctcttcttttggatgtcagactccaagatatctatcatcatgaatcgtgatcaaactttg

SEQ ID NO: 4 - DNA, 150 bp, Artificial
  DNA encoding artificial miRNA precursor
catgagggttaagctatttcagttgaatcgttattacctctgtcaaaggatattatagatatatacatgtgtatgttaatgattcaagtgatcatagagagtatcctttgacagaggtaataacgatccaacatgttattgcctctgatc

SEQ ID NO: 5 - RNA, 226 nt, Artificial
  Artificial miRNA precursor
guguaacagaaucguuauuaccucugucaaaacgaagaggacaagauuugaggaacuaaaaugcagaaucuaagaguucaugucuuccucauagagagugcgcgguguuaaaagcuugaagaaagcacacuuuaaggggauugcacgaccucuuagauucucccucuuucucuacauaucauucucuucucuucguuugacagagguaauaacgauuuucuucuuc

SEQ ID NO: 6 - RNA, 226 nt, Artificial
  Arificial miRNA precursor
gaagaagaaaaucguuauuaccucugucaaacgaagagaagagaaugauauguagagaaagagggagaaucuaagaggucgugcaauccccuuaaagugugcuuucuucaagcuuuuaacaccgcgcacucucuaugaggaagacaugaacucuuagauucugcauuuuaguuccucaaaucuuguccucuucguuuugacagagguaauaacgauucuguuacac

SEQ ID NO: 7 - RNA, 150 nt, Artificial
  Artificial miRNA precursor
caugaggguuaagcuauuucaguugaaucguuauuaccucugucaaaggauauuauagauauauacauguguauguuaaugauucaagugaucauagagaguauccuuugacagagguaauaacgauccaacauguuauugccucugauc

SEQ ID NO: 8 - RNA, 150 nt, Artificial
  Artificial miRNA precursor
gaucagaggcaauaacauguuggaucguuauuaccucugucaaaggauacucucuaugaucacuugaaucauuaacauacacauguauauaucuauaauauccuuugacagagguaauaacgauucaacugaaauagcuuaacccucaug

SEQ ID NO: 9 - RNA, 222 nt, Artificial
  Arificial miRNA precursor
aaugaugauuacgaugaugagagucucuaguuguaucagagggucuugcauggaagaauagagaaugagguugaguuuagucugacuuggccaguuuuuuuaccaaugaaucuaauuaacugauucugguguuggccaagucagacuaaacucuguuuccuucucuucuuuuggaugucagacuccaagauaucuaucaucaugaaucgugaucaaacuuug

SEQ ID NO: 10 - RNA, 222 nt, Artificial
  Arificial miRNA precursor
caaaguuugaucacgauucaugaugauagauaucuuggagucugacauccaaaagaagagaaggaaacagaguuuagucugacuuggccaacaccagaaucaguuaauuagauucauugguaaaaaaacuggccaagucagacuaaacucaaccucauucucuauucuuccaugcaagacccucugauacaacuagagacucucaucaucguaaucaucauu

SEQ ID NO: 11 - RNA, 273 nt, Artificial
  Arificial miRNA precursor
acaguuugcuuaugucggauccauaauauauuugacaagauacuuuguuuuucgauagaucuugaucugacgauggaagcagucgagugcgugagcaagucaugaguugagcaggguaaagaaaagcugcuaagcuauggaucccauaagcccuaauccuuguaaaguaaaaaaggauuugguuauauggauugcauaucucaggagcuuuaacuugcccuuuaauggcuuuuacucuucacuugcucacgcacucgacugcaucccggguca

SEQ ID NO: 12 - RNA, 273 nt, Artificial
  Artificial miRNA precursor
ugacccgggaugcagucgagugcgugagcaagugaagaguaaaagccauuaaagggcaaguuaaagcuccugagauaugcaauccauauaaccaaauccuuuuuuacuuuacaaggauuagggcuuaugggauccauagcuuagcagcuuuucuuuacccugcucaacucaugacuugcucacgcacucgacugcuuccaucgucagaucaagaucuaucgaaaaacaaaguaucuugucaaauauauuauggauccgacauaagcaaacugu

SEQ ID NO: 13 - RNA, 21 nt, Artificial
  Artificial miRNA
gucagcucacgcacucguuca

(19) United States
(12) Patent Application Publication
(10) Pub. No.: US 2008/0313773 A1
(43) Pub. Date: Dec. 18, 2008

(54) PRODUCTION OF ARTIFICIAL MICRORNAS USING SYNTHETIC MICRORNA PRECURSORS

(75) Inventors: Nam-Hai CHUA, New York, NY (US); Qi-Wen NIU, New York, NY (US)

Correspondence Address: ROTHWELL, FIGG, ERNST & MANBECK, P.C., 1425 K STREET, N.W., SUITE 800, WASHINGTON, DC, 20005, US

(73) Assignee: THE ROCKEFELLER UNIVERSITY, New York, NY (US)

(21) Appl. No.: 12/117,095

(22) Filed: May 8, 2008

Related U.S. Application Data
(60) Provisional application No. 60/917,756, filed on May 14, 2007.

(51) Int. Cl. A01H 1/00; C07H 21/02; C12N 5/04; A01H 5/10; C12N 15/82; A01H 5/00; C12Q 1/68

(52) U.S. Cl. 800/278; 536/23.1; 435/419; 800/298; 435/468; 435/6

(57) ABSTRACT

The invention provides methods and compositions useful in target sequence suppression, target sequence validation and target sequence down regulation. The invention provides polynucleotide constructs useful for producing artificial microRNA (amiRNA) using synthetic amiRNA precursors.

### CROSS-REFERENCE TO RELATED APPLICATIONS

The present application is related to and claims priority under 35 U.S.C. § 119(e) to U.S. provisional patent application Ser. No. 60/917,756 filed 14 May 2007, which is incorporated herein by reference.

### BACKGROUND OF THE INVENTION

The field of the present invention relates generally to plant molecular biology and plant biotechnology. More specifically, it relates to constructs for the production of artificial microRNA (amiRNA) using synthetic amiRNA precursors. The constructs can be used in methods to suppress the expression of targeted genes or to down regulate targeted genes.

The publications and other materials used herein to illuminate the background of the invention, and in particular, cases to provide additional details respecting the practice, are incorporated by reference, and for convenience are referenced in the following text.

RNA interference refers to the process of sequence-specific post-transcriptional gene silencing in animals mediated by short interfering RNAs (siRNAs) (Fire et al. (1998) Nature 391:806-811). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire (1999) Trends Genet. 15:358-363). Such protection from foreign gene expression may have evolved in response to the production of double-stranded RNAs (dsRNAs) derived from viral infection or from the random integration of transposon elements into a host genome via a cellular response that specifically destroys homologous single-stranded RNA of viral genomic RNA. The presence of dsRNA in cells triggers the RNAi response through a mechanism that has yet to be fully characterized.

Recently, microRNAs (miRNAs) have been identified as important regulators of gene expression in both plants and animals. miRNAs are single-stranded RNAs, 20-24 nucleotides (nt) in length, generated from processing of longer pre-miRNA precursors (Bartel (2004 Cell 116:281-297) by DCL1 in Arabidopsis thaliana (Xie et al. (2004) PLoS Biol 2:642-652). These miRNAs are recruited to the RISC complex. Using RNA:RNA base-pairing, miRNAs direct RISC in a sequence-specific manner to downregulate target mRNAs in one of two ways. Limited miRNA:mRNA base-pairing results in translational repression, which is the case with the majority of the animal miRNAs studied so far. By contrast, most plant miRNAs show extensive base-pairing to, and guide cleavage of, their target mRNAs (Jones-Rhoades et al. (2006) Annu Rev Plant Biol 57:19-53; Llave et al. (2002) Proc Natl Acad Sci USA 97:13401-13406). In A. thaliana, miRNAs are known to be important regulators of plant developmental processes. Previous reports have shown that alteration of several nucleotides within an miRNA 21-nt sequence does not affect its biogenesis (Vauchert et al. (2004) Genes Dev 18:1187-1197).

Plant pre-miRNAs can be redesigned by replacing the 21-nt mature miRNA sequence, as well as the miRNA complementary sequence (the miRNA* strand or miRNA star strand), with 21-nt synthetic sequences. Such artificial pre-miRNAs have sequences identical to those of the natural pre-miRNAs except in the region encoding the mature miRNA and its star strand. By this method, artificial miRNAs (amiRNA) have been designed that can target and silence specific transcripst with complementary sequences. Different amiRNAs have successfully been produced using the backbone of pre-miR159a, pre-miR159c, pre-miR169a and pre-miR169g. Moreover, it has been shown that transgenic Arabidopsis plants expressing amiRNAs with sequences complementary to those of RNA viruses can be rendered resistant or even immune to these viruses. (Niu et al. (2006) Nat Biotechnol 24:1420-1428). Other reports (Schwab et al. (2006) Plant Cell 18:1121-1133; Alvarez et al. (2006) Plant Cell 18:1134-1151) also indicated that amiRNAs can be used to down regulate endogenous plant genes. However, in all these successful examples, native miRNA precursors (pre-miR159a, miR164b, miR172a and miR319a) were used as backbones to generate artificial miRNAs. So far, there are no reports on the production of amiRNAs using a synthetic miRNA precursor.

### SUMMARY OF THE INVENTION

The present invention relates to constructs for the production of artificial microRNA (amiRNA) using synthetic amiRNA precursors. The constructs can be used in methods to suppress the expression of targeted genes or to down regulate targeted genes. The present invention also relates to methods for screening synthetic amiRNA precursors that can be used for the production of amiRNAs. In accordance with the present invention, amiRNAs are generated from a totally new synthetic amiRNA precursor without using the native miRNA precursor as a backbone. The synthetic amiRNA precursor is the antisense strand of a miRNA precursor, which antisense strand is never used as a template for transcription in plants. The use of the antisense strand is an easy and efficient way to generate synthetic amiRNA precursors. In addition to producing amiRNAs for target gene suppression, the use of the antisense strand provides a new method to study the relationships of amiRNA precursor and/or miRNA precursor folding structure and processing efficiency.

Thus, in one aspect the present invention provides synthetic amiRNA precursors and constructs containing the synthetic amiRNA precursors. In one embodiment, the synthetic amiRNA precursor is an antisense strand of a native, i.e., naturally occurring miRNA precursor, which has been modified to contain a desired amiRNA sequence and a sequence complementary to the desired amiRNA. The desired amiRNA is substantially complementary to a target sequence and is heterologous to the native miRNA precursor. In one embodiment, the native miRNA sequences have been replaced by the desired amiRNA sequences. The synthetic amiRNA precursor is capable of forming a double-stranded RNA or a hairpin. The synthetic amiRNA precursor comprises the desired amiRNA and a sequence complementary to the desired amiRNA, wherein the desired amiRNA is an amiRNA modified to be (i) fully complementary to the target sequence or (ii) fully complementary to the target sequence except for GU base pairing.

In one embodiment, the nucleic acid construct comprises a polynucleotide encoding a synthetic amiRNA precursor. In another embodiment, the nucleic acid construct comprises more than one polynucleotide each encoding different synthetic amiRNA precursors. As is well known in the art, the amiRNA precursor forms a hairpin which in some cases the double-stranded region may be very short, e.g., not exceeding 21-25 bp in length. In one embodiment, the nucleic acid construct may further comprise a promoter operably linked to the polynucleotide. The promoter initiates and mediates transcription of the antisense strand of the amiRNA precursor, i.e., initiates and mediates transcription of the synthetic amiRNA precursor. In another embodiment, the nucleic acid construct may further comprise separate promoters each operably linked to a different one of the polynucleotides. In this embodiment, the separate promoters may be the same or different. In a further embodiment, the nucleic acid construct may further comprise a single promoter operatively linked to all of the polynucleotides. The promoter may be a pathogen-inducible promoter or other inducible promoters. The binding of the amiRNA to the target RNA leads to cleavage of the target RNA. The target sequence of a target RNA may be a coding sequence, an intron or a splice site.

In a second aspect, the present invention provides a method of producing an amiRNA in a cell. In one embodiment, the method comprises the steps of:

(a) introducing into a cell a nucleic acid construct comprising a promoter operably linked to a polynucleotide which encodes an artificial miRNA (amiRNA) precursor capable of forming a double-stranded RNA or a hairpin, wherein the amiRNA precursor comprises a modified miRNA precursor in which the miRNA sequence and its complementary (miRNA* strand) sequence are replaced by an amiRNA sequence and its fully complementary sequence, respectively, wherein the amiRNA sequence is (i) fully complementary to a target sequence, (ii) fully complementary to the target sequence except for GU base pairing or (iii) fully complementary to the target sequence in the first ten nucleotides counting from the 5′ end of the amiRNA, wherein the promoter initiates and mediates transcription of the antisense strand of the amiRNA precursor and

(b) growing the cell under conditions wherein the promoter initiates and mediates transcription of the amiRNA precursor and the amiRNA is produced.

In a second embodiment, the method comprises the steps of:

(a) introducing into a cell a polynucleotide which encodes an artificial miRNA (amiRNA) precursor capable of forming a double-stranded RNA or a hairpin, wherein the amiRNA precursor comprises a modified miRNA precursor in which the miRNA sequence and its complementary (miRNA* strand) sequence are replaced by an amiRNA sequence and its fully complementary sequence, respectively, wherein the amiRNA sequence is (i) fully complementary to a target sequence, (ii) fully complementary to the target sequence except for GU base pairing or (iii) fully complementary to the target sequence in the first ten nucleotides counting from the 5′ end of the amiRNA, wherein the polynucleotide is operably linked to a host cell promoter, and wherein the host cell promoter initiates and mediates transcription of the antisense strand of the amiRNA precursor and

(b) growing the cell under conditions wherein the promoter initiates and mediates transcription of the antisense strand of the amiRNA precursor and the amiRNA is produced.

In another embodiment, the method comprises the steps of:

(a) identifying a precursor miRNA containing a miRNA sequence and a complementary miRNA sequence (a miRNA* strand sequence),

(b) replacing the miRNA sequence with an amiRNA sequence and replacing the miRNA complementary sequence (the miRNA* strand sequence) with a complementary sequence of the amiRNA sequence that is substantially complementary to produce an amiRNA precursor;

(c) introducing the amiRNA precursor or a nucleic acid construct containing the amiRNA precursor into a cell; and

(d) growing the cell under conditions wherein the promoter initiates and mediates transcription of the amiRNA precursor and the amiRNA is produced.

In a third aspect, the present invention provides a method for determining synthetic amiRNA precursors that can be used for the production of amiRNAs. i.e., a method to assay for the production of amiRNAs. In one embodiment, the method to assay for production of an amiRNA comprises the steps of:

(a) identifying a precursor miRNA containing a miRNA sequence and a complementary miRNA sequence (a miRNA* strand sequence),

(b) introducing into a cell a nucleic acid construct comprising a promoter operably linked to a polynucleotide encoding an artificial miRNA (amiRNA) precursor wherein the amiRNA precursor comprises the following:
(1) an artificial miRNA sequence replacing the endogenous miRNA sequence;
(2) a fully complementary sequence of the artificial miRNA replacing the endogenous miRNA* strand sequence;

wherein the promoter initiates and mediates transcription of the antisense strand of the amiRNA precursor;

(c) growing the cell of (b) under conditions wherein the promoter initiates and mediates transcription of the antisense strand of the amiRNA precursor; and

(e) assaying for production of the amiRNA.

In another embodiment, the amiRNA precursor of step (b) further comprises (3) replacement of one or more potential GU base pairs in the predicted secondary structure of the amiRNA precursor with one or more CG or AT base pairs.

In another embodiment, the amiRNA precursor of step (b) further comprises (3) replacement of one or more potential GC base pairs in the predicted secondary structure of the amiRNA precursor with one or more AT base pairs.

In another embodiment, the amiRNA precursor of step (b) further comprises (3) replacement of one or more potential AT base pairs in the predicted secondary structure of the amiRNA precursor with one or more CG base pairs.

According to another aspect, the present invention provides a vector that can be used to express pre-amiRNAs or pre-miRNAs in both the sense and antisense direction. The vector is useful for screening candidate sequences for new synthetic amiRNA precursors. The vector is also useful for comparing expression levels of different synthetic amiRNA precursors with different folding structures by agroinfiltration. In one embodiment, the vector comprises a T-DNA binary vector with two cloning sites. A first cloning site is downstream of a first plant operable promoter and is a multiple restriction site. A second cloning site is downstream of a second plant operable promoter and is a Gateway cassette (Hartley et al. (2000) Genome Res 10:1788-1795; Earley et al. (2006) The Plant J 45:616-629). In one embodiment, the first promoter is a CaMV 35S promoter. In another embodiment, the second promoter is a synthetic G10-90 promoter.

According to a further aspect, the present invention provides a cell comprising the polynucleotide or nucleic acid construct of the present invention. In some embodiments, the polynucleotide or nucleic acid construct of the present invention may be inserted into an intron of a gene or a transgene of the cell. The cell may be a plant cell, either a monocot or a dicot, including, but not limited to, corn, wheat, rice, barley, oats, sorghum, millet, sunflower, safflower, cotton, soy, canola, alfalfa, Arabidopsis, and tobacco.

According to another aspect, the present invention provides a transgenic plant comprising the isolated polynucleotide or nucleic acid construct. In some embodiments, the isolated polynucleotide or nucleic acid construct of the present invention may be inserted into an intron of a gene or a transgene of the transgenic plant. The transgenic plant may be either a monocot or a dicot, including, but not limited to, corn, wheat, rice, barley, oats, sorghum, millet, sunflower, safflower, cotton, soy, canola, alfalfa, Arabidopsis, and tobacco.

According to another aspect, the present invention relates to the use of synthetic pre-miRNAs to generate mature miRNAs in transformants to down regulate any target gene or target genes. Synthetic pre-miRNAs include modified native pre-miRNAs, antisense native pre-miRNAs, antisense modified native pre-miRNAs and artificial designed pre-miRNAs. Mature miRNAs include native mature miRNAs and amiRNAs. Transformants refer to transformed cells (i.e., transformed plant cells, transformed non-human animal cells, transformed human cells in vitro, transformed animal stem cells or transformed human stem cells), transformed plants and transformed non-human animals.

### BRIEF DESCRIPTION OF THE FIGURES

FIGS. 1A-1D show the sequences of four pre-amiRNAs, pre-amiR-HC-Pro^{159a} (SEQ ID NO:1), pre-amiR-Fhy1^{159c} (SEQ ID NO:2), pre-amiR-PDS^{169g} (SEQ ID NO:3) and pre-amiR-Fhy1^{165a} (SEQ ID NO:4). Mature amiRNAs and its reverse complementary sequence (amiRNA* strand) are in bold italic and bold, respectively. The remaining nucleotide sequences are native pre-miRNAs backbone sequences.

FIG. 2 shows a schematic of vector pDCS, a T-DNA transformation binary vector containing two cloning sites. One cloning site is multiple restriction enzyme site downstream of a CaMV 35S promoter. The second cloning site is a Gateway cassette placed downstream of the synthetic G10-90 promoter.

FIGS. 3A-D show the predicted folding structures of the 4 sense and anti-sense pre-amiRNAs. Sequences of the mature amiRNA are in bold italic. pre-amiR-Fhy1^{159c}: SEQ ID NO:5; pre-amiR-Fhy1^{159c}A: SEQ ID NO:6; pre-amiR-Fhy1^{165a}: SEQ ID NO:7; pre-amiR-Fhy1^{165a}A: SEQ ID NO:8; pre-amiR-PDS^{169g}: SEQ ID NO:9; pre-amiR-PDS^{169g}A: SEQ ID NO:10; pre-amiR-HC-Pro^{159a}: SEQ ID NO:11; pre-amiR-HC-Pro^{159a}A: SEQ ID NO:12.

FIGS. 4a-4d show that pre-amiR-HC-Pro^{159a}A and pre-amiR-Fhy1^{159c}A were able to generate amiRNAs (FIGS. 4a, 4b) whereas pre-amiR-PDS^{169g}A appeared unable to generate amiR-PDS^{169g} (FIG. 4c) and the same was found for pre-amiR-Fhy1^{165a}A (data not shown). FIG. 4d: Northern blot results are shown.

FIG. 5 shows that the processing of anti-sense pre-miRNAs requires DCL1. As shown in this figure, Northern blot analysis indicated that both endogenous and artificial miRNAs levels were clearly decreased in dcll-9 mutant as compared to WT plants. For example, miR-169, an endogenous miRNA, was not detectable in dcll-9 mutants and in transgenic plants showing dcll-9 phenotype (pre-miR-HC-P^{159a}A dcll-9 or pre-miR-P69^{159a}A dcll-9). On the other hand, high miR-169 expression levels were detected in WT and transgenic plants showing WT phenotype (pre-miR-HC-P^{159a}A WT or pre-miR-P69^{159a}A WT). Levels of matured artificial miRNAs (amiR-HC-P^{159a} or amiR-P69^{159a}) in dcll-9 mutant transgenic plants (pre-miR-HC-P^{159a}A dcll-9 or pre-miR-P69^{159a}A dcll-9) were considerably lower than in WT transgenic plants (pre-miR-HC-P^{159a}A wt or pre-miR-P69^{159a}A wt).

FIGS. 6a and 6b show that expression levels of amiR-HC-P^{159a} vary for lines expressing the pre-amiR-HC-P^{159a}A construct with line#4 and line#6 having a lower and a higher level, respectively. Similar variations in amiR-P69^{159a} expression levels were found amongst transgenic lines expressing pre-amiR-P69^{159a}A. As expected, no amiR-HC-P^{159a} nor amiR-P69^{159a} was detected in non-transgenic WT plants. Notwithstanding the variation in amiRNA expression levels there were no significant differences in endogenous miR-159a levels amongst WT and these different transgenic lines (FIG. 6a and FIG. 6b).

FIGS. 7a and 7b show amiRNA expression levels of independent transgenic lines. As expected, there was wide variation in the amiRNA expression level amongst transgenic lines.

FIGS. 8a and 8b show that WT plants were sensitive to TuMV infection displaying lesions on systemic leaves whereas T1 transgenic plants expressing pre-amiR-HC-P^{159a}A did not show any symptoms. FIG. 8a: TYMV infection. FIG. 8b: TuMV infection.

### DETAILED DESCRIPTION OF THE INVENTION

The present invention relates to constructs for the production of artificial microRNA (amiRNA) using synthetic amiRNA precursors. The constructs can be used in methods to suppress the expression of targeted genes or to down regulate targeted genes. The present invention also relates to methods for determining synthetic amiRNA precursors that can be used for the production of amiRNAs.

The invention provides methods and compositions useful for suppressing targeted sequences. The compositions can be employed in any type of plant cell, and in other cells which comprise the appropriate processing components (e.g., RNA interference components), including invertebrate and vertebrate animal cells. The compositions and methods are based on an endogenous miRNA silencing process discovered in Arabidopsis, a similar strategy can be used to extend the number of compositions and the organisms in which the methods are used. The methods can be adapted to work in any eukaryotic cell system. Additionally, the compositions and methods described herein can be used in individual cells, cells or tissue in culture, or in vivo in organisms, or in organs or other portions of organisms.

The compositions selectively suppress the target sequence by encoding an amiRNA having substantial complementarity to a region of the target sequence. The amiRNA is provided in a nucleic acid construct which, when transcribed into RNA, is predicted to form a hairpin structure which is processed by the cell to generate the amiRNA, which then suppresses expression of the target sequence.

A nucleic acid construct is provided to encode the amiRNA for any specific target sequence. Any amiRNA can be inserted into the construct, such that the encoded amiRNA selectively targets and suppresses the target sequence.

A method for suppressing a target sequence is provided. The method employs the constructs above, in which an amiRNA is designed to a region of the target sequence, and inserted into the construct. Upon introduction into a cell, the amiRNA produced suppresses expression of the targeted sequence. The target sequence can be an endogenous plant sequence, or a heterologous transgene in the plant. The target gene may also be a gene from a plant pathogen, such as a pathogenic virus, nematode, insect, or mold or fungus.

A plant, cell, and seed comprising the construct and/or the amiRNA is provided. Typically, the cell will be a cell from a plant, but other eukaryotic cells are also contemplated, including but not limited to yeast, insect, nematode, or animal cells. Plant cells include cells from monocots and dicots. The invention also provides plants and seeds comprising the construct and/or the amiRNA. Viruses and prokaryotic cells comprising the construct are also provided.

Units, prefixes, and symbols may be denoted in their SI accepted form. Unless otherwise indicated, nucleic acids are written left to right in 5′ to 3′ orientation; amino acid sequences are written left to right in amino to carboxyl orientation, respectively. Numeric ranges recited within the specification are inclusive of the numbers defining the range and include each integer within the defined range. Amino acids may be referred to herein by either commonly known three letter symbols or by the one-letter symbols recommended by the IUPAC-IUB Biochemical Nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5^{th} edition, 1993). The terms defined below are more fully defined by reference to the specification as a whole.

As used herein, “artificial miRNA” or “amiRNA” refers to a small oligoribonucleic acid, typically about 19-25 nucleotides in length, that is not a naturally occurring miRNA, and which suppresses expression of a polynucleotide comprising the target sequence transcript or down regulates a target RNA. An amiRNA is typically produced using an amiRNA precursor.

As used herein, an “amiRNA precursor” refers to a larger polynucleotide which is different from native pre-miRNA and able to produce a mature amiRNA. This larger polynucleotide includes a DNA which encodes an RNA precursor, and an RNA transcript comprising the amiRNA. The amiRNA precursor is typically a modified native miRNA precursor. An amiRNA precursor is sometimes also referred to as a pre-amiRNA.

As used herein, a “mature amiRNA” refers to the amiRNA generated from the processing of an amiRNA precursor.

As used herein, an “amiRNA template” is an oligonucleotide region, or regions, in a nucleic acid construct or a polynucleotide which encodes the amiRNA.

As used herein, the “amiRNA* strand” or “amiRNA star strand” is a portion of a polynucleotide or a nucleic acid construct which is substantially complementary to the amiRNA template and is predicted to base pair with the amiRNA template. The amiRNA template and amiRNA* strand may form a double-stranded polynucleotide, including a hairpin structure. As is known for natural miRNAs, the mature amiRNA and its complements may contain mismatches and form bulges and thus do not need to be fully complementary. The “amiRNA* strand” may also sometimes be referred to as the “backside” region of an amiRNA.

As used herein, a “modified native miRNA precursor” refers to a native miRNA precursor that has been modified by replacing the native miRNA with the desired amiRNA or replacing some nucleotides by others or removing some parts of a native pre-miRNA or adding some designed RNA to a native pre-miRNA.

As used herein, “native miRNA precursor” refers to a miRNA precursor that is naturally occurring in plants.

As used herein, “synthetic amiRNA precursor” refers to an amiRNA precursor that contains RNA sequence outside the amiRNA region that is not found in a naturally occurring RNA. It includes modified native miRNA precursors, antisense native miRNA precursors, antisense modified native miRNA precursors and artificial designed pre-miRNAs.

As used herein, “nucleic acid construct” or “construct” refers to an isolated polynucleotide which is introduced into a host cell. This construct may comprise any combination of deoxyribonucleotides, ribonucleotides, and/or modified nucleotides. The construct may be transcribed to form an RNA, wherein the RNA may be capable of forming a double-stranded RNA and/or hairpin structure. This construct may be expressed in the cell, or isolated or synthetically produced. The construct may further comprise a promoter, or other sequences which facilitate manipulation or expression of the construct.

As used here “suppression” or “silencing” or “inhibition” are used interchangeably to denote the down-regulation of the expression of the product of a target sequence relative to its normal expression level in a wild type organism. Suppression includes expression that is decreased by about 10%, 15%, 20%, 25%, 30%, 35%, 40%, 45%, 50%, 55%, 60%, 65%, 70%, 75%, 80%, 85%, 90%, 95%, or 100% relative to the wild type expression level.

As used herein, “encodes” or “encoding” with respect to a DNA sequence refers to a DNA sequence which can provides the template to generate an RNA, and when the RNA is an mRNA, the corresponding polypeptide.

As used herein, “expression” or “expressing” refers to the generation of an RNA transcript from an introduced construct, an endogenous DNA sequence, or a stably incorporated heterologous DNA sequence. The term may also refer to a polypeptide produced from an mRNA generated from any of the above DNA precursors.

As used herein, “heterologous” in reference to a nucleic acid is a nucleic acid that originates from a foreign species, or is synthetically designed, or, if from the same species, is substantially modified from its native form in composition and/or genomic locus by deliberate human intervention. A heterologous protein may originate from a foreign species or, if from the same species, is substantially modified from its original form by deliberate human intervention.

By “host cell” is meant a cell which contains an introduced nucleic acid construct and supports the replication and/or expression of the construct. Host cells may be prokaryotic cells such as E. coli, or eukaryotic cells such as fungi, yeast, insect, amphibian, nematode, or mammalian cells. Alternatively, the host cells are monocotyledonous or dicotyledonous plant cells. An example of a monocotyledonous host cell is a maize host cell.

The term “introduced” means providing a nucleic acid or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing.

The term “isolated” refers to material, such as a nucleic acid or a protein, which is: (1) substantially or essentially free from components which normally accompany or interact with the material as found in its naturally occurring environment or (2) if the material is in its natural environment, the material has been altered by deliberate human intervention to a composition and/or placed at a locus in the cell other than the locus native to the material.

As used herein, the phrases “target sequence” and “sequence of interest” are used interchangeably. Target sequence is used to mean the nucleic acid sequence that is selected for suppression of expression, and is not limited to polynucleotides encoding polypeptides. The target sequence comprises a sequence that is substantially or completely complementary to the amiRNA. The target sequence can be RNA or DNA, and may also refer to a polynucleotide comprising the target sequence.

As used herein, “nucleic acid” means a polynucleotide and includes single or double-stranded polymer of deoxyribonucleotide or ribonucleotide bases. Nucleic acids may also include fragments and modified nucleotides.

By “nucleic acid library” is meant a collection of isolated DNA or RNA molecules which comprise and substantially represent the entire transcribed fraction of a genome of a specified organism or of a tissue from that organism. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, Calif. (Berger); Sambrook et al., Molecular Cloning—A Laboratory Manual, 2nd ed., Vol. 1-3 (1989); and Current Protocols in Molecular Biology, F. M. Ausubel et al., Eds., Current Protocols, a joint venture between Greene Publishing Associates, Inc. and John Wiley & Sons, Inc. (1994).

As used herein “operably linked” includes reference to a functional linkage of at least two sequences. Operably linked includes linkage between a promoter and a second sequence, wherein the promoter sequence initiates and mediates transcription of the DNA sequence corresponding to the second sequence.

As used herein, “plant” includes plants and plant parts including but not limited to plant cells, plant tissue such as leaves, stems, roots, flowers, and seeds.

As used herein, “polypeptide” means proteins, protein fragments, modified proteins, amino acid sequences and synthetic amino acid sequences. The polypeptide can be glycosylated or not.

As used herein, “promoter” includes reference to a region of DNA that is involved in recognition and binding of an RNA polymerase and other proteins to initiate transcription.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to and including 100% sequence identity (i.e., fully complementary) with each other.

The term “stringent conditions” or “stringent hybridization conditions” includes reference to conditions under which a probe will selectively hybridize to its target sequence. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50° to 55° C. Exemplary moderate stringency conditions include hybridization in 40 to 45% formamide, 1 M NaCl, 1% SDS at 37° C., and a wash in 0.5× to 1×SSC at 55° to 60° C. Exemplary high stringency conditions include hybridization in 50% formamide, 1 M NaCl, 1% SDS at 37° C., and a wash in 0.1×SSC at 60° to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl ((1984) Anal Biochem 138:267-284): Tₘ=81.5° C.+16.6 (log M)+0.41 (% GC)−0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6°, 7°, 8°, 9°, or 10° C. lower than the thermal melting point (Tₘ); low stringency conditions can utilize a hybridization and/or wash at 11°, 12°, 13°, 14 °, 15°, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y. (1993); and Current Protocols in Molecular Biology, Chapter 2, Ausubel et al., Eds., Greene Publishing and Wiley-Interscience, New York (1995). Hybridization and/or wash conditions can be applied for at least 10, 30, 60, 90, 120, or 240 minutes.

As used herein, “transgenic” includes reference to a plant or a cell which comprises a heterologous polynucleotide. Generally, the heterologous polynucleotide is stably integrated within the genome such that the polynucleotide is passed on to successive generations. Transgenic is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, “vector” includes reference to a nucleic acid used in introduction of a polynucleotide of the invention into a host cell. Expression vectors permit transcription of a nucleic acid inserted therein.

Polynucleotide sequences may have substantial identity or substantial complementarity to the selected region of the target gene. As used herein “substantial identity” indicates sequences that have sequence identity or homology to each other. Generally, sequences that are substantially identical will have at least about 75%, 80%, 85%, 90%, 91%, 92%, 93%, 94%, 95%, 96%, 97%, 98%, 99%, or 100% sequence identity wherein the percent sequence identity is based on the entire sequence and is determined by GAP alignment using default parameters (GCG, GAP version 10, Accelrys, San Diego, Calif.). GAP uses the algorithm of Needleman and Wunsch ((1970) J Mol Biol 48:443-453) to find the alignment of two complete sequences that maximizes the number of matches and minimizes the number of sequence gaps. Sequences which have 100% identity are identical. “Substantial complementarity” refers to sequences that are complementary to each other, and are able to base pair with each other. In describing complementary sequences, if all the nucleotides in the first sequence will base pair to the second sequence, these sequences are fully complementary. If additionally, the first and second sequence have the same number of nucleotides, then each sequence is the “full complement” or “full-length complement” of the other.

In its broadest sense, the present invention relates to the use of synthetic pre-miRNAs to generate mature miRNAs in transformants to down regulate any target gene or target genes. Synthetic pre-miRNAs include modified native pre-miRNAs, antisense native pre-miRNAs, antisense modified native pre-miRNAs and artificial designed pre-miRNAs. Mature miRNAs include native mature miRNAs and amiRNAs. Transformants refer to transformed cells (i.e., transformed plant cells, transformed non-human animal cells, transformed human cells in vitro, transformed animal stem cells or transformed human stem cells), transformed plants and transformed non-human animals.

In one embodiment, there is provided a method for the suppression of a target sequence comprising introducing into a cell a nucleic acid construct encoding an amiRNA substantially complementary to the target. In some embodiments the amiRNA comprises about 10-200 nucleotides, about 10-15, 15-20, 19, 20, 21, 22, 23, 24, 25, 26, 27, 25-30, 30-50, 50-100, 100-150, or about 150-200 nucleotides. In some embodiments the nucleic acid construct encodes a polynucleotide precursor which may form a double-stranded RNA, or hairpin structure comprising the amiRNA.

In some embodiments, the nucleic acid construct comprises a promoter that mediates transcription of the antisense strand of a modified native plant miRNA precursor, wherein the precursor has been modified to replace the native miRNA encoding regions with sequences designed to produce an amiRNA directed to the target sequence. In one embodiment, the nucleic acid construct comprises a promoter that mediates transcription of the antisense strand of a modified miR159a miRNA precursor. In another embodiment, the nucleic acid construct comprises a promoter that mediates transcription of the antisense strand of a modified miR159c miRNA precursor.

In another embodiment, the method comprises selecting a target sequence of a gene, and designing a nucleic acid construct comprising a polynucleotide encoding an amiRNA substantially complementary to the target sequence. In some embodiments, the target sequence is selected from any region of the gene. In some embodiments, the target sequence is selected from an untranslated region. In some embodiments, the target sequence is selected from a coding region of the gene. In some embodiments, the target sequence is selected from a region which overlaps a coding and a non-coding region of the RNA. In some embodiments, the target sequence is selected from a region about 50 to about 200 nucleotides upstream from the stop codon, including regions from about 50-75, 75-100, 100-125, 125-150, or 150-200 upstream from the stop codon.

In some embodiments, the amiRNA template, (i.e. the polynucleotide encoding the amiRNA), and thereby the amiRNA, may comprise some mismatches relative to the target sequence. In some embodiments the amiRNA template has ≧1 nucleotide mismatch as compared to the target sequence, for example, the amiRNA template can have 1, 2, 3, 4, 5, or more mismatches as compared to the target sequence. This degree of mismatch may also be described by determining the percent identity of the amiRNA template to the complement of the target sequence. For example, the amiRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the target sequence.

In some embodiments, the amiRNA template, (i.e. the polynucleotide encoding the amiRNA) and thereby the amiRNA, may comprise some mismatches relative to the formation of a duplex with miRNA backside. In some embodiments the amiRNA template has ≧1 nucleotide mismatch as compared to the miRNA backside, for example, the amiRNA template can have 1, 2, 3, 4, 5, or more mismatches as compared to the amiRNA backside. This degree of mismatch may also be described by determining the percent identity of the amiRNA template to the complement of the amiRNA backside. For example, the amiRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the amiRNA backside.

In some embodiments, the target sequence is selected from a plant pathogen. Plants or cells comprising an amiRNA directed to the target sequence of the pathogen are expected to have decreased sensitivity and/or increased resistance to the pathogen. In some embodiments, the amiRNA is encoded by a nucleic acid construct further comprising an operably linked promoter. In some embodiments, the promoter is a pathogen-inducible promoter.

In another embodiment a method is provided comprising a method of inhibiting expression of a target sequence in a cell. That is, the method provides for the down regulation of an RNA containing a target sequence. In one embodiment, the method comprises:

(a) introducing into a cell a nucleic acid construct comprising a promoter operably linked to a polynucleotide which encodes an artificial miRNA (amiRNA) precursor capable of forming a double-stranded RNA or a hairpin, wherein the amiRNA precursor comprises a modified miRNA precursor in which the miRNA sequence and its complementary (miRNA* strand) sequence are replaced by an amiRNA sequence and its fully complementary sequence, respectively, wherein the amiRNA sequence is (i) fully complementary to a target sequence, (ii) fully complementary to the target sequence except for GU base pairing or (iii) fully complementary to the target sequence in the first ten nucleotides counting from the 5′ end of the amiRNA, wherein the promoter initiates and mediates transcription of the antisense strand of the amiRNA precursor and

(b) growing the cell of (a) under conditions wherein the promoter initiates and mediates transcription of the antisense strand of the amiRNA precursor, the amiRNA is produced and the RNA containing the target sequence is cleaved.

In a second embodiment, the method comprises:

(a) introducing into a cell a polynucleotide which encodes an artificial miRNA (amiRNA) precursor capable of forming a double-stranded RNA or a hairpin, wherein the amiRNA precursor comprises a modified miRNA precursor in which the miRNA sequence and its complementary (miRNA* strand) sequence are replaced by an amiRNA sequence and its fully complementary sequence, respectively, wherein the amiRNA sequence is (i) fully complementary to a target sequence, (ii) fully complementary to the target sequence except for GU base pairing or (iii) fully complementary to the target sequence in the first ten nucleotides counting from the 5′ end of the amiRNA, wherein the polynucleotide is operably linked to a host cell promoter, and wherein the host cell promoter initiates and mediates transcription of the antisense strand of the amiRNA precursor and

(b) growing the cell of (a) under conditions wherein the promoter initiates and mediates transcription of the antisense strand of the amiRNA precursor, the amiRNA is produced and the RNA containing the target sequence is cleaved.

In a third embodiment, the method comprises the steps:

(a) identifying a precursor miRNA containing a miRNA sequence and a complementary miRNA sequence (a miRNA* strand sequence),

(b) replacing the miRNA sequence with an amiRNA sequence to produce an amiRNA precursor and replacing the miRNA complementary sequence (the miRNA* strand sequence) with a complementary sequence of the amiRNA sequence that is substantially complementary to produce an amiRNA precursor;

(c) introducing the amiRNA precursor or a nucleic acid construct containing the amiRNA precursor into a cell; and

(d) expressing the antisense strand of the amiRNA precursor to produce the amiRNA, wherein the amiRNA inhibits expression of the target sequence.

In another embodiment, there is provided a polynucleotide that encodes an amiRNA that is substantially complementary to the target. In some embodiments, the polynucleotide encodes a synthetic amiRNA precursor. In some embodiments, the amiRNA is encoded by the antisense strand of an amiRNA precursor.

In another embodiment, there is provided a method of producing an amiRNA in a cell. In one embodiment, the method comprises the steps of:

(a) introducing into a cell a nucleic acid construct comprising a promoter operably linked to a polynucleotide which encodes an artificial miRNA (amiRNA) precursor capable of forming a double-stranded RNA or a hairpin, wherein the amiRNA precursor comprises a modified miRNA precursor in which the miRNA sequence and its complementary (miRNA* strand) sequence are replaced by an amiRNA sequence and its fully complementary sequence, respectively, wherein the amiRNA sequence is (i) fully complementary to a target sequence, (ii) fully complementary to the target sequence except for GU base pairing or (iii) fully complementary to the target sequence in the first ten nucleotides counting from the 5′ end of the amiRNA, wherein the promoter initiates and mediates transcription of the antisense strand of the amiRNA precursor and

(b) growing the cell under conditions wherein the promoter initiates and mediates transcription of the amiRNA precursor and the amiRNA is produced.

In a second embodiment, the method comprises the steps of:

(a) introducing into a cell a polynucleotide which encodes an artificial miRNA (amiRNA) precursor capable of forming a double-stranded RNA or a hairpin, wherein the amiRNA precursor comprises a modified miRNA precursor in which the miRNA sequence and its complementary (miRNA* strand) sequence are replaced by an amiRNA sequence and its fully complementary sequence, respectively, wherein the amiRNA sequence is (i) fully complementary to a target sequence, (ii) fully complementary to the target sequence except for GU base pairing or (iii) fully complementary to the target sequence in the first ten nucleotides counting from the 5′ end of the amiRNA, wherein the polynucleotide is operably linked to a host cell promoter, and wherein the host cell promoter initiates and mediates transcription of the antisense strand of the amiRNA precursor and (b) growing the cell under conditions wherein the promoter initiates and mediates transcription of the antisense strand of the amiRNA precursor and the amiRNA is produced.

In another embodiment, the method comprises the steps of:

(a) identifying a precursor miRNA containing a miRNA sequence and a complementary miRNA sequence (a miRNA* strand sequence),

(b) replacing the miRNA sequence with an amiRNA sequence to produce an amiRNA precursor and replacing the miRNA complementary sequence (the miRNA* strand sequence) with a complementary sequence of the amiRNA sequence that is substanially complementary to produce an amiRNA precursor;

(c) introducing the amiRNA precursor or a nucleic acid construct containing the amiRNA precursor into a cell; and

(d) expressing the antisense strand of the amiRNA precursor to produce the amiRNA.

In another embodiment, there is provided a method for determining synthetic amiRNA precursors that can be used for the production of amiRNAs. i.e., a method to assay for the production of amiRNAs. In this manner, amiRNA precursors are screened to identify those useful for producing amiRNAs. In one embodiment, the method to assay for production of an amiRNA comprises the steps of:

(a) identifying a precursor miRNA containing a miRNA sequence and a complementary miRNA sequence (a miRNA* strand sequence),

(b) introducing into a cell a nucleic acid construct comprising a promoter operably linked to a polynucleotide encoding an artificial miRNA (amiRNA) precursor wherein the amiRNA precursor comprises the following:

(1) an artificial miRNA sequence replacing the endogenous miRNA sequence;

(2) a fully complementary sequence of the artificial miRNA replacing the endogenous miRNA* strand sequence; and

wherein the promoter initiates and mediates transcription of the antisense strand of the amiRNA precursor;

(c) growing the cell of (b) under conditions wherein the promoter initiates and mediates transcription of the antisense strand of the amiRNA precursor; and

(d) assaying for production of the artificial miRNA.

In another embodiment, the amiRNA precursor of step (b) further comprises (3) replacement of one or more potential GU base pairs in the predicted secondary structure of the amiRNA precursor with one or more CG or AT base pairs.

In another embodiment, the amiRNA precursor of step (b) further comprises (3) replacement of one or more potential GC base pairs in the predicted secondary structure of the amiRNA precursor with one or more AT base pairs.

In another embodiment, the amiRNA precursor of step (b) further comprises (3) replacement of one or more potential AT base pairs in the predicted secondary structure of the amiRNA precursor with one or more CG base pairs.

In another embodiment, there is provided a nucleic acid construct for suppressing a target sequence. The nucleic acid construct comprises a polynucleotide that encodes an amiRNA substantially complementary to the target. In some embodiments, the nucleic acid construct further comprises a promoter operably linked to the polynucleotide encoding the amiRNA. The promoter initiates and mediates transcription of the antisense strand of the amiRNA precursor, i.e., initiates and mediates transcription of the synthetic amiRNA precursor. In some embodiments, the nucleic acid construct lacking a promoter is designed and introduced in such a way that it becomes operably linked to a promoter upon integration in the host genome. The host promoter initiates and mediates transcription of the antisense strand of the amiRNA precursor, i.e., initiates and mediates transcription of the synthetic amiRNA precursor. In some embodiments, the nucleic acid construct is integrated using recombination, including site-specific recombination. See, for example, PCT International published application No. WO 99/25821, incorporated herein by reference. In some embodiments, the nucleic acid construct is an RNA. In some embodiments, the nucleic acid construct comprises at least one recombination site, including site-specific recombination sites. In some embodiments the nucleic acid construct comprises at least one recombination site in order to facilitate integration, modification, or cloning of the construct. In some embodiments the nucleic acid construct comprises two site-specific recombination sites flanking the synthetic amiRNA precursor. In some embodiments the site-specific recombination sites include FRT sites, lox sites, or att sites, including attB, attL, attP or attR sites. See, for example, PCT International published application No. WO 99/25821, and U.S. Pat. Nos. 5,888,732, 6,143,557, 6,171,861, 6,270,969, and 6,277,608, each incorporated herein by reference.

In some embodiments, the nucleic acid construct comprises a promoter that initiates and mediates transcription of the antisense strand of a modified native plant miRNA precursor, wherein the precursor has been modified to replace the miRNA encoding region with a sequence designed to produce an amiRNA directed to the target sequence. In one embodiment, the nucleic acid construct comprises a promoter that initiates and mediates transcription of the antisense strand of a modified miR159a miRNA precursor. In another embodiment, the nucleic acid construct comprises a promoter that initiates and mediates transcription of the antisense strand of a modified miR159c miRNA precursor.

In another embodiment, the nucleic acid construct comprises a promoter that initiates and mediates transcription of an isolated polynucleotide comprising a polynucleotide which is the antisense strand of a modified plant miRNA precursor, the modified precursor comprising a first and a second oligonucleotide, wherein at least one of the first and second oligonucleotides is heterologous to the precursor, wherein the first oligonucleotide is substantially complementary to the second oligonucleotide, and wherein the second oligonucleotide comprises an amiRNA substantially complementary to the target sequence, wherein the precursor is capable of forming a hairpin.

In another embodiment, the nucleic acid construct comprises a promoter that initiates and mediates transcription of an isolated polynucleotide comprising a polynucleotide which is the antisense strand of a modified plant miR159a plant miRNA precursor, the modified precursor comprising a first and a second oligonucleotide, wherein at least one of the first and second oligonucleotides is heterologous to the precursor, wherein the first oligonucleotide is substantially complementary to the second oligonucleotide, and wherein the second oligonucleotide comprises an amiRNA substantially complementary to the target sequence, wherein the precursor is capable of forming a hairpin. In some embodiments, the modified plant miR159a miRNA precursor is a modified Arabidopsis miR159a miRNA precursor, or a modified corn miR159a miRNA precursor, or a modified rice miR159a miRNA precursor, or the like.

In another embodiment, the nucleic acid construct comprises a promoter that initiates and mediates transcription of an isolated polynucleotide comprising a polynucleotide which is the antisense strand of a modified plant miR159c plant miRNA precursor, the modified precursor comprising a first and a second oligonucleotide, wherein at least one of the first and second oligonucleotides is heterologous to the precursor, wherein the first oligonucleotide is substantially complementary to the second oligonucleotide, and wherein the second oligonucleotide comprises an amiRNA substantially complementary to the target sequence, wherein the precursor is capable of forming a hairpin. In some embodiments, the modified plant miR159c miRNA precursor is a modified Arabidopsis miR159c miRNA precursor, or a modified corn miR159c miRNA precursor, or a modified rice miR159c miRNA precursor, or the like.

In some embodiments the amiRNA comprises about 10-200 nucleotides, about 10-15, 15-20, 19, 20, 21, 22, 23, 24, 25, 26, 27, 25-30, 30-50, 50-100, 100-150, or about 150-200 nucleotides. In some embodiments the nucleic acid construct encodes a polynucleotide precursor which may form a double-stranded RNA, or hairpin structure comprising the amiRNA. In some embodiments, the target region is selected from any region of the target sequence. In some embodiments, the target region is selected from an untranslated region. In some embodiments, the target region is selected from a coding region of the target sequence. In some embodiments, the target sequence is selected from a region which overlaps a coding and a non-coding region of the RNA. In some embodiments, the target region is selected from a region about 50 to about 200 nucleotides upstream from the stop codon, including regions from about 50-75, 75-100, 100-125, 125-150, or 150-200 upstream from the stop codon.

In some embodiments, there are provided cells, plants, and seeds comprising the introduced polynucleotides, and/or produced by the methods of the invention. The cells include prokaryotic and eukaryotic cells, including but not limited to bacteria, yeast, fungi, viral, invertebrate, vertebrate, and plant cells. Plants, plant cells, and seeds of the invention include gynosperms, monocots and dicots, including but not limited to, for example, corn (maize), rice, wheat, oats, barley, millet, sorghum, soy, sunflower, safflower, canola, alfalfa, cotton, Arabidopsis, and tobacco.

In some embodiments, the cells, plants, and/or seeds comprise a nucleic acid construct comprising a promoter that initiates and mediates transcription of the antisense strand of a modified plant miRNA precursor, wherein the precursor has been modified to replace the native miRNA encoding regions with sequences designed to produce an amiRNA directed to the target sequence. In some embodiments, the miRNA precursor template is a miR159a miRNA precursor or a miR159c miRNA precursor. In some embodiments, the miR159a miRNA precursor or a miR159c miRNA precursor is from a dicot or a monocot. In some embodiments, the miR159a miRNA precursor or a miR159c miRNA precursor is from Arabidopsis thaliana, tomato, soybean, rice, or corn. In some embodiments, the nucleic acid construct comprises at least one recombination site, including site-specific recombination sites. In some embodiments, the nucleic acid construct comprises at least one recombination site in order to facilitate modification or cloning of the construct. In some embodiments, the nucleic acid construct comprises two site-specific recombination sites flanking the miRNA precursor. In some embodiments, the site-specific recombination sites include FRT sites, lox sites, or att sites, including attB, attL, attP or attR sites. See, for example, PCT International published application No. WO 99/25821, and U.S. Pat. Nos. 5,888,732, 6,143,557, 6,171,861, 6,270,969, and 6,277,608, herein incorporated by reference.

In a further embodiment, there is provided a method for down regulating a target RNA comprising introducing into a cell a nucleic acid construct that encodes an amiRNA that is complementary to a region of the target RNA. In some embodiments, the amiRNA is fully complementary to the region of the target RNA. In some embodiments, the amiRNA is complementary and includes the use of G-U base pairing, i.e. the GU wobble, to otherwise be fully complementary. In some embodiments, the first ten nucleotides of the amiRNA (counting from the 5′ end of the amiRNA) are fully complementary to a region of the target RNA and the remaining nucleotides may include mismatches and/or bulges with the target RNA. In some embodiments the amiRNA comprises about 10-200 nucleotides, about 10-15, 15-20, 19, 20, 21, 22, 23, 24, 25, 26, 27, 25-30, 30-50, 50-100, 100-150, or about 150-200 nucleotides. In some embodiments, the binding of the amiRNA to the complementary sequence in the target RNA results in cleavage of the target RNA. In some embodiments, the binding of the amiRNA to the complementary sequence in the target RNA results in translational repression. In some embodiments, the target sequence is selected from a region which overlaps a coding and a non-coding region of the RNA. In some embodiments, the amiRNA is a miRNA that has been modified such that the amiRNA is fully complementary to the target sequence of the target RNA. In some embodiments, the amiRNA is a native plant miRNA that has been modified such that the amiRNA is fully complementary to the target sequence of the target RNA. In some embodiments, the polynucleotide encoding the amiRNA is operably linked to a promoter. In some embodiments, the nucleic acid construct comprises a promoter operably linked to the amiRNA. The promoter initiates and mediates transcription of the antisense strand of the amiRNA precursor, i.e., initiates and mediates transcription of the synthetic amiRNA precursor.

In some embodiments, the nucleic acid construct encodes the amiRNA. In some embodiments, the nucleic acid construct comprises a promoter operably linked to the amiRNA. The promoter initiates and mediates transcription of the antisense strand of the amiRNA precursor, i.e., initiates and mediates transcription of the synthetic amiRNA precursor. In some embodiments, the nucleic acid construct encodes a polynucleotide which may form a double-stranded RNA, or hairpin structure comprising the amiRNA. In some embodiments, the nucleic acid construct comprises a promoter operably linked to the polynucleotide which may form a double-stranded RNA, or hairpin structure comprising the amiRNA. The promoter initiates and mediates transcription of the antisense strand of the amiRNA precursor, i.e., initiates and mediates transcription of the synthetic amiRNA precursor. In some embodiments, the nucleic acid construct comprises a native plant miRNA precursor that has been modified such that the amiRNA is fully complementary to the target sequence of the target RNA. In some embodiments, the nucleic acid construct comprises a promoter operably linked to the amiRNA precursor. The promoter initiates and mediates transcription of the antisense strand of the amiRNA precursor, i.e., initiates and mediates transcription of the synthetic amiRNA precursor. In some embodiments, the nucleic acid construct comprises about 50 nucleotides to about 3000 nucleotides, about 50-100, 100-150, 150-200, 200-250, 250-300, 300-350, 350-400, 400-450, 450-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2500, 2500-2600, 2600-2700, 2700-2800, 2800-2900 or about 2900-3000 nucleotides.

In some embodiments, the nucleic acid construct lacking a promoter is designed and introduced in such a way that it becomes operably linked to a promoter upon integration in the host genome. The host promoter initiates and mediates transcription of the antisense strand of the amiRNA precursor, i.e., initiates and mediates transcription of the synthetic amiRNA precursor. In some embodiments, the nucleic acid construct is integrated using recombination, including site-specific recombination. In some embodiments, the nucleic acid construct is an RNA. In some embodiments, the nucleic acid construct comprises at least one recombination site, including site-specific recombination sites. In some embodiments the nucleic acid construct comprises at least one recombination site in order to facilitate integration, modification, or cloning of the construct. In some embodiments the nucleic acid construct comprises two site-specific recombination sites flanking the amiRNA precursor.

In another embodiment, the method comprises a method for down regulating a target RNA in a cell comprising introducing into the cell a nucleic acid construct that encodes an amiRNA that is complementary to a region of the target RNA and expressing the nucleic acid construct for a time sufficient to produce amiRNA, wherein the amiRNA down regulates the target RNA. In some embodiments, the amiRNA is fully complementary to the region of the target RNA. In some embodiments, the amiRNA is complementary and includes the use of G-U base pairing, i.e. the GU wobble, to otherwise be fully complementary.

In another embodiment, the method comprises selecting a target RNA, selecting an miRNA, comparing the sequence of the target RNA (or its DNA) with the sequence of the miRNA, identifying a region of the target RNA (or its DNA) in which the nucleotide sequence is similar to the nucleotide sequence of the miRNA, modifying the nucleotide sequence of the miRNA so that it is complementary to the nucleotide sequence of the identified region of the target RNA to produce an amiRNA and preparing a nucleic acid construct comprising the amiRNA. In some embodiments, the amiRNA is fully complementary to the identified region of the target RNA. In some embodiments, the amiRNA is complementary and includes the use of G-U base pairing, i.e. the GU wobble, to otherwise be fully complementary. In some embodiments, a nucleic acid construct encodes a polynucleotide which may form a double-stranded RNA, or hairpin structure comprising the amiRNA. In some embodiments, a nucleic acid construct comprises a precursor of the amiRNA, i.e., a pre-miRNA that has been modified in accordance with this embodiment.

In another embodiment, the method comprises selecting a target RNA, selecting a nucleotide sequence within the target RNA, selecting a miRNA, modifying the sequence of the miRNA so that it is complementary to the nucleotide sequence of the identified region of the target RNA to produce an amiRNA and preparing a nucleic acid construct comprising the amiRNA. In some embodiments, the amiRNA is fully complementary to the identified region of the target RNA. In some embodiments, the amiRNA is complementary and includes the use of G-U base pairing, i.e. the GU wobble, to otherwise be fully complementary. In some embodiments, a nucleic acid construct encodes a polynucleotide which may form a double-stranded RNA, or hairpin structure comprising the amiRNA. In some embodiments, a nucleic acid construct comprises a precursor of the amiRNA, i.e., a pre-miRNA that has been modified in accordance with this embodiment.

In some embodiments, the miRNA is a miRNA disclosed in the microRNA registry, now also known as the miRBase Sequence Database (Griffiths-Jones (2004) Nucl Acids Res 32, Database issue:D109-D111; http:// microrna dot sanger dot ac dot uk/). In some embodiments, the miRNA is ath-MIR156a, ath-MIR156b, ath-MIR156c, ath-MIR156d, ath-MIR156e, ath-MIR156f, ath-MIR156g, ath-MIR156h, ath-MIR157a, ath-MIR157b, ath-MIR157c, ath-MIR157d, ath-MIR158a, ath-MIR158b, ath-MIR159a, ath-MIR159b, ath-MIR159c, ath-MIR160a, ath-MIR160b, ath-MIR160c, ath-MIR161, ath-MIR162a, ath-MIR162b, ath-MIR163, ath-MIR164a, ath-MIR164b, ath-MIR164c, ath-MIR165a, ath-MIR165b, ath-MIR166a, ath-MIR166b, ath-MIR166c, ath-MIR166d, ath-MIR166e, ath-MIR166f, ath-MIR166g, ath-MIR167a, ath-MIR167b, ath-MIR167c, ath-MIR167d, ath-MIR168a, ath-MIR168b, ath-MIR169a, ath-MIR169b, ath-MIR169c, ath-MIR169d, ath-MIR169e, ath-MIR169f, ath-MIR169g, ath-MIR169h, ath-MIR169i, ath-MIR169j, ath-MIR169k, ath-MIR1691, ath-MIR169m, ath-MIR169n, ath-MIR170, ath-MIR171a, ath-MIR171b, ath-MIR171c, ath-MIR172a, ath-MIR172b, ath-MIR172c, ath-MIR172d, ath-MIR172e, ath-MIR173, ath-MIR319a, ath-MIR319b, ath-MIR319c, ath-MIR390a, ath-MIR390b, ath-MIR391, ath-MIR393a, ath-MIR393b, ath-MIR394a, ath-MIR394b, ath-MIR395a, ath-MIR395b, ath-MIR395c, ath-MIR395d, ath-MIR395e, ath-MIR395f, ath-MIR396a, ath-MIR396b, ath-MIR397a, ath-MIR397b, ath-MIR398a, ath-MIR398b, ath-MIR398c, ath-MIR399a, ath-MIR399b, ath-MIR399c, ath-MIR399d, ath-MIR399e, ath-MIR399f, ath-MIR400, ath-MIR401, ath-MIR402, ath-MIR403, ath-MIR404, ath-MIR405a, ath-MIR405b, ath-MIR405d, ath-MIR406, ath-MIR407, ath-MIR408, ath-MIR413, ath-MIR414, ath-MIR415, ath-MIR416, ath-MIR417, ath-MIR418, ath-MIR419, ath-MIR420, ath-MIR426, ath-MIR447a, ath-MIR447b, ath-MIR447c, ath-MIR472, ath-MIR771, ath-MIR773, ath-MIR773, ath-MIR774, ath-MIR775, ath-MIR776, ath-MIR777, ath-MIR778, ath-MIR779, ath-MIR780, ath-MIR781, ath-MIR782, ath-MIR783, ath-MIR822, ath-MIR823, ath-MIR824, ath-MIR825, ath-MIR826, ath-MIR827, ath-MIR828, ath-MIR829, ath-MIR830, ath-MIR831, ath-MIR832, ath-MIR833, ath-MIR834, ath-MIR835, ath-MIR836, ath-MIR837, ath-MIR838, ath-MIR839, ath-MIR840, ath-MIR841, ath-MIR842, ath-MIR843, ath-MIR844, ath-MIR845a, ath-MIR845b, ath-MIR846, ath-MIR847, ath-MIR848, ath-MIR849, ath-MIR850, ath-MIR851, ath-MIR852, ath-MIR853, ath-MIR854a, ath-MIR854b, ath-MIR854c, ath-MIR854d, ath-MIR855, ath-MIR856, ath-MIR857, ath-MIR858, ath-MIR859, ath-MIR860, ath-MIR861, ath-MIR862, ath-MIR863, ath-MIR864, ath-MIR865, ath-MIR866, ath-MIR867, ath-MIR868, ath-MIR869, ath-MIR870.

In some embodiments, the miRNA is osa-MIR156a, osa-MIR156b, osa-MIR156c, osa-MIR156d, osa-MIR156e, osa-MIR156f, osa-MIR156g, osa-MIR156h, osa-MIR156i, osa-MIR156j, osa-MIR156k, osa-MIR156l, osa-MIR159a, osa-MIR159b, osa-MIR159c, osa-MIR159d, osa-MIR159e, osa-MIR159f, osa-MIR160a, osa-MIR160b, osa-MIR160c, osa-MIR160d, osa-MIR160e, osa-MIR160f, osa-MIR162a, osa-MIR162b, osa-MIR164a, osa-MIR164b, osa-MIR164c, osa-MIR164d, osa-MIR164e, osa-MIR164f, osa-MIR166a, osa-MIR166b, osa-MIR166c, osa-MIR166d, osa-MIR166e, osa-MIR166f, osa-MIR166g, osa-MIR166h, osa-MIR166i, osa-MIR166j, osa-MIR166k, osa-MIR166l, osa-MIR166m, osa-MIR166n, osa-MIR167a, osa-MIR167b, osa-MIR167c, osa-MIR167d, osa-MIR167e, osa-MIR167f, osa-MIR167g, osa-MIR167h, osa-MIR167i, osa-MIR167j, osa-MIR168a, osa-MIR168b, osa-MIR169a, osa-MIR169b, osa-MIR169c, osa-MIR169d, osa-MIR169e, osa-MIR169f, osa-MIR169g, osa-MIR169h, osa-MIR169i, osa-MIR169j, osa-MIR169k, osa-MIR169l, osa-MIR169m, osa-MIR169n, osa-MIR169o, osa-MIR169p, osa-MIR169q, osa-MIR171a, osa-MIR171b, osa-MIR171c, osa-MIR171d, osa-MIR171e, osa-MIR171f, osa-MIR171g, osa-MIR171h, osa-MIR171i, osa-MIR172a, osa-MIR172b, osa-MIR172c, osa-MIR172d, osa-MIR319a, osa-MIR319b, osa-MIR390, osa-MIR393, osa-MIR393b, osa-MIR394, osa-MIR395a, osa-MIR395b, osa-MIR395c, osa-MIR395d, osa-MIR395e, osa-MIR395f, osa-MIR395g, osa-MIR395h, osa-MIR395i, osa-MIR395j, osa-MIR395k, osa-MIR395l, osa-MIR395m, osa-MIR395n, osa-MIR395o, MIR395p, osa-MIR395q, osa-MIR395r, osa-MIR395s, osa-MIR395t, osa-MIR395u, osa-MIR395v, osa-MIR395w, osa-MIR396a, osa-MIR396b, osa-MIR396c, osa-MIR396d, osa-MIR396e, osa-MIR397a, osa-MIR397b, osa-MIR398a, osa-MIR398b, osa-MIR399a, osa-MIR399b, osa-MIR399c, osa-MIR399d, osa-MIR399e, osa-MIR399f, osa-MIR399g, osa-MIR399h, osa-MIR399i, osa-MIR399j, osa-MIR399k, osa-MIR408, osa-MIR413, osa-MIR414, osa-MIR415, osa-MIR416, osa-MIR417, osa-MIR418, osa-MIR419, osa-MIR420, osa-MIR426, osa-MIR435, osa-MIR437, osa-MIR438, osa-MIR439a, osa-MIR439b, osa-MIR439c, osa-MIR439d, osa-MIR439e, osa-MIR439f, osa-MIR439g, osa-MIR439h, MIR439i, MIR439j, osa-MIR440, osa-MIR441a, osa-MIR441b, osa-MIR441c, osa-MIR442, osa-MIR443, osa-MIR444, osa-MIR445a, osa-MIR445b, osa-MIR445c, osa-MIR445d, osa-MIR445e, osa-MIR445f, osa-MIR445g, osa-MIR445h, osa-MIR445i, osa-MIR446, osa-MIR528, osa-MIR529, osa-MIR530, osa-MIR531, osa-MIR535, osa-MIR806a, osa-MIR806b, osa-MIR806c, osa-MIR806d, osa-MIR806e, osa-MIR806f, osa-MIR806g, osa-MIR806h, osa-MIR807a, osa-MIR807b, osa-MIR807c, osa-MIR808, osa-MIR809a, osa-MIR 809b, osa-MIR809c, osa-MIR809d, osa-MIR809e, osa-MIR809f, osa-MIR809g, osa-MIR 809h, osa-MIR810, osa-MIR811a, osa-MIR811b, osa-MIR811c, osa-MIR812a, osa-MIR812b, osa-MIR812c, osa-MIR812d, osa-MIR812e, osa-MIR813, osa-MIR814a, osa-MIR814b, osa-MIR814c, osa-MIR815a, osa-MIR815b, osa-MIR815c, osa-MIR816, osa-MIR817, osa-MIR818a, osa-MIR818b, osa-MIR818c, osa-MIR818d, osa-MIR818e, osa-MIR819a, osa-MIR819b, osa-MIR819c, osa-MIR819d, osa-MIR819e, osa-MIR819f, osa-MIR819g, osa-MIR819h, osa-MIR819i, osa-MIR819j, osa-MIR819k, osa-MIR820a, osa-MIR820b, osa-MIR820c, osa-MIR821a, osa-MIR821b, osa-MIR821c.

In some embodiments, the miRNA is zma-MIR156a, zma-MIR156b, zma-MIR156c, zma-MIR156d, zma-MIR156e, zma-MIR156f, zma-MIR156g, zma-MIR156h, zma-MIR156i, zma-MIR156j, zma-MIR156k, zma-MIR159a, zma-MIR159b, zma-MIR159c, zma-MIR159d, zma-MIR160a, zma-MIR160b, zma-MIR160c, zma-MIR160d, zma-MIR160e, zma-MIR160f, zma-MIR162, zma-MIR164a, zma-MIR164b, zma-MIR164c, zma-MIR164d, zma-MIR166a, zma-MIR166b, zma-MIR166c, zma-MIR166d, zma-MIR166e, zma-MIR166f, zma-MIR166g, zma-MIR166h, zma-MIR166i, zma-MIR166j, zma-MIR166k, zma-MIR1661, zma-MIR166m, zma-MIR167a, zma-MIR167b, zma-MIR167c, zma-MIR167d, zma-MIR167e, zma-MIR167f, zma-MIR167g, zma-MIR167h, zma-MIR167i, zma-MIR168a, zma-MIR168b, zma-MIR169a, zma-MIR169b, zma-MIR169c, zma-MIR169d, zma-MIR169e, zma-MIR169f, zma-MIR169g, zma-MIR169h, zma-MIR169i, zma-MIR169j, zma-MIR169k, zma-MIR171a, zma-MIR171b, zma-MIR171c, zma-MIR171d, zma-MIR171e, zma-MIR171f, zma-MIR171g, zma-MIR171h, zma-MIR171i, zma-MIR171j, zma-MIR171k, zma-MIR172a, zma-MIR172b, zma-MIR172c or zma-MIR172d, zma-MIR172e, zma-MIR319a, zma-MIR319b, zma-MIR319c, zma-MIR319d, zma-MIR393, zma-MIR394a, zma-MIR394b, zma-MIR395a, zma-MIR395b, zma-MIR395c, zma-MIR396a, zma-MIR396b, zma-MIR399a, zma-MIR399b, zma-MIR399c, zma-MIR399d, zma-MIR399e, zma-MIR399f, zma-MIR408.

In some embodiments, the miRNA is gma-MIR156a, gma-MIR156b, gma-MIR156c, gma-MIR156d, gma-MIR156e, gma-MIR159, gma-MIR160, gma-MIR166a, gma-MIR166b, gma-MIR167a, gma-MIR167b, gma-MIR168, gma-MIR169, gma-MIR172a, gma-MIR172b, gma-MIR319a, gma-MIR319b, gma-MIR319c, gma-MIR396a, gma-MIR396b, gma-MIR398a, gma-MIR398b.

In some embodiments, the miRNA is mtr-MIR156, mtr-MIR160, mtr-MIR162, mtr-MIR166, mtr-MIR169a, mtr-MIR169b, mtr-MIR171, mtr-MIR319, mtr-MIR393, mtr-MIR395a, mtr-MIR395b, mtr-MIR395 c, mtr-MIR395 d, mtr-MIR395 e, mtr-MIR395 f, mtr-MIR395g, mtr-MIR395h, mtr-MIR395i, mtr-MIR395j, mtr-MIR395k, mtr-MIR3951, mtr-MIR395m, mtr-MIR395n, mtr-MIR395o, mtr-MIR395p, mtr-MIR399a, mtr-MIR399b, mtr-MIR399c, mtr-MIR399d, mtr-MIR399e.

In some embodiments, the miRNA is ppt-MIR156a, ppt-MIR319a, ppt-MIR319b, ppt-MIR319c, ppt-MIR319d, ppt-MIR319a, ppt-MIR390a, ppt-MIR390b, ppt-MIR390c, ppt-MIR533a, ppt-MIR533b, ppt-MIR534, ppt-MIR535a, ppt-MIR535b, ppt-MIR535c, ppt-MIR535d, ppt-MIR536, ppt-MIR537a, ppt-MIR537b, ppt-MIR538a, ppt-MIR538b, ppt-MIR538c, ppt-MIR1210, ppt-MIR1211, ppt-MIR1212, ppt-MIR1213, ppt-MIR1214, ppt-MIR1215, ppt-MIR1216, ppt-MIR1217, ppt-MIR1218, ppt-MIR1219a, ppt-MIR1219b, ppt-MIR1219c, ppt-MIR1219d, ppt-MIR1220a, ppt-MIR1220b, ppt-MIR1221, ppt-MIR1222, ppt-MIR1223.

In some embodiments, the miRNA is ptc-MIR156a, ptc-MIR156b, ptc-MIR156c, ptc-MIR156d, ptc-MIR156e, ptc-MIR156f, ptc-MIR156g, ptc-MIR156h, ptc-MIR156i, ptc-MIR156j, ptc-MIR156k, ptc-MIR159a, ptc-MIR159b, ptc-MIR159c, ptc-MIR159d, ptc-MIR159e, ptc-MIR159f, ptc-MIR160a, ptc-MIR160b, ptc-MIR160c, ptc-MIR160d, ptc-MIR160e, ptc-MIR160f, ptc-MIR160g, ptc-MIR160h, ptc-MIR162a, ptc-MIR162b, ptc-MIR162c, ptc-MIR164a, ptc-MIR164b, ptc-MIR164c, ptc-MIR164d, ptc-MIR164e, ptc-MIR164f, ptc-MIR166a, ptc-MIR166b, ptc-MIR166c, ptc-MIR166d, ptc-MIR166e, ptc-MIR166f, ptc-MIR166g, ptc-MIR166h, ptc-MIR166i, ptc-MIR166j, ptc-MIR166k, ptc-MIR1661, ptc-MIR166m, ptc-MIR166n, ptc-MIR166o, ptc-MIR166p, ptc-MIR166q, ptc-MIR167a, ptc-MIR167b, ptc-MIR167c, ptc-MIR167d, ptc-MIR167e, ptc-MIR167f, ptc-MIR167g, ptc-MIR167h, ptc-MIR168a, ptc-MIR168b, ptc-MIR169a, ptc-MIR169aa, ptc-MIR169ab, ptc-MIR169ac, ptc-MIR169ad, ptc-MIR169ae, ptc-MIR169af, ptc-MIR169b, ptc-MIR169c, ptc-MIR169d, ptc-MIR169e, ptc-MIR169f, ptc-MIR169g, ptc-MIR169h, ptc-MIR169i, ptc-MIR169j, ptc-MIR169k, ptc-MIR1691, ptc-MIR169m, ptc-MIR169n, ptc-MIR169o, ptc-MIR169p, ptc-MIR169q, ptc-MIR169r, ptc-MIR169s, ptc-MIR169t, ptc-MIR169u, ptc-MIR169v, ptc-MIR169w, ptc-MIR169x, ptc-MIR169y, ptc-MIR169z, ptc-MIR171a, ptc-MIR171b, ptc-MIR171c, ptc-MIR171d, ptc-MIR171e, ptc-MIR171f, ptc-MIR171g, ptc-MIR171h, ptc-MIR171i, ptc-MIR171j, ptc-MIR171k, ptc-MIR172a, ptc-MIR172b, ptc-MIR172c, ptc-MIR172d, ptc-MIR172e, ptc-MIR172f, ptc-MIR172g, ptc-MIR172h, ptc-MIR172i, ptc-MIR319a, ptc-MIR319b, ptc-MIR319c, ptc-MIR319d, ptc-MIR319e, ptc-MIR319f, ptc-MIR319g, ptc-MIR319h, ptc-MIR319i, ptc-MIR390a, ptc-MIR390b, ptc-MIR390c, ptc-MIR390d, ptc-MIR393a, ptc-MIR393b, ptc-MIR393c, ptc-MIR393d, ptc-MIR394a, ptc-MIR394b, ptc-MIR395a, ptc-MIR395b, ptc-MIR395c, ptc-MIR395d, ptc-MIR395e, ptc-MIR395f, ptc-MIR395g, ptc-MIR395h, ptc-MIR395i, ptc-MIR395j, ptc-MIR396a, ptc-MIR396b, ptc-MIR396c, ptc-MIR396d, ptc-MIR396e, ptc-MIR396f, ptc-MIR396g, ptc-MIR397a, ptc-MIR397b, ptc-MIR397c, ptc-MIR398a, ptc-MIR398b, ptc-MIR398c, ptc-MIR399a, ptc-MIR399b, ptc-MIR399c, ptc-MIR399d, ptc-MIR399e, ptc-MIR399f, ptc-MIR399g, ptc-MIR399h, ptc-MIR399i, ptc-MIR399j, ptc-MIR399k, ptc-MIR3991, ptc-MIR403a, ptc-MIR403b, ptc-MIR403c, ptc-MIR408, ptc-MIR472a, ptc-MIR472b, ptc-MIR473a, ptc-MIR473b, ptc-MIR474a, ptc-MIR474b, ptc-MIR474c, ptc-MIR475a, ptc-MIR475b, ptc-MIR475c, ptc-MIR475d, ptc-MIR476a, ptc-MIR476b, ptc-MIR476c, ptc-MIR477a, ptc-MIR477b, ptc-MIR478a, ptc-MIR478b, ptc-MIR478c, ptc-MIR478d, ptc-MIR478e, ptc-MIR478f, ptc-MIR478h, ptc-MIR478i, ptc-MIR478j, ptc-MIR478k, ptc-MIR4781, ptc-MIR478m, ptc-MIR478n, ptc-MIR478o, ptc-MIR478p, ptc-MIR478q, ptc-MIR478r, ptc-MIR478s, ptc-MIR478u, ptc-MIR479, ptc-MIR480a, ptc-MIR480b, ptc-MIR481a, ptc-MIR481b, ptc-MIR481c, ptc-MIR481d, ptc-MIR481e, ptc-MIR482.

In some embodiments, the miRNA is sof-MIR156, sof-MIR159a, sof-MIR159b, sof-MIR159c, sof-MIR159d, sof-MIR159e, sof-MIR167a, sof-MIR167b, sof-MIR168a, sof-MIR168b, sof-MIR396, sof-MIR408a, sof-MIR408b, sof-MIR408c, sof-MIR408d, sof-MIR408e.

In some embodiments, the miRNA is sbi-MIR156a, sbi-MIR156b, sbi-MIR156c, sbi-MIR156d, sbi-MIR156e, sbi-MIR159, sbi-MIR159b, sbi-MIR160a, sbi-MIR160b, sbi-MIR160c, sbi-MIR160d, sbi-MIR160e, sbi-MIR164, sbi-MIR164b, sbi-MIR164c, sbi-MIR166a, sbi-MIR166b, sbi-MIR166c, sbi-MIR166d, sbi-MIR166e, sbi-MIR166f, sbi-MIR166g, sbi-MIR167a, sbi-MIR167b, sbi-MIR167c, sbi-MIR167d, sbi-MIR167e, sbi-MIR167f, sbi-MIR167g, sbi-MIR168, sbi-MIR169a, sbi-MIR169b, sbi-MIR169c, sbi-MIR169d, sbi-MIR169e, sbi-MIR169f, sbi-MIR169g, sbi-MIR169h, sbi-MIR169i, sbi-MIR171a, sbi-MIR171b, sbi-MIR171c, sbi-MIR171d, sbi-MIR171e, sbi-MIR171f, sbi-MIR172a, sbi-MIR172b, sbi-MIR172c, sbi-MIR172d, sbi-MIR172e, sbi-MIR319, sbi-MIR393, sbi-MIR394a, sbi-MIR394b, sbi-MIR395 a, sbi-MIR395b, sbi-MIR395 c, sbi-MIR395 d, sbi-MIR395 e, sbi-MIR395f, sbi-MIR396a, sbi-MIR396b, sbi-MIR396c, sbi-MIR399a, sbi-MIR399b, sbi-MIR399c, sbi-MIR399d, sbi-MIR399e, sbi-MIR399f, sbi-MIR399g, sbi-MIR399h, sbi-MIR399i.

In some embodiments, the miRNA is a miRNA disclosed in Genbank (USA), EMBL (Europe) or DDBJ (Japan). In some embodiments, the miRNA is selected from one of the following Genbank accession numbers: AJ505003, AJ505002, AJ505001, AJ496805, AJ496804, AJ496803, AJ496802, AJ496801, AJ505004, AJ493656, AJ493655, AJ493654, AJ493653, AJ493652, AJ493651, AJ493650, AJ493649, AJ493648, AJ493647, AJ493646, AJ493645, AJ493644, AJ493643, AJ493642, AJ493641, AJ493640, AJ493639, AJ493638, AJ493637, AJ493636, AJ493635, AJ493634, AJ493633, AJ493632, AJ493631, AJ493630, AJ493629, AJ493628, AJ493627, AJ493626, AJ493625, AJ493624, AJ493623, AJ493622, AJ493621, AJ493620, AY615374, AY615373, AY730704, AY730703, AY730702, AY730701, AY730700, AY730699, AY730698, AY599420, AY551259, AY551258, AY551257, AY551256, AY551255, AY551254, AY551253, AY551252, AY551251, AY551250, AY551249, AY551248, AY551247, AY551246, AY551245, AY551244, AY551243, AY551242, AY551241, AY551240, AY551239, AY551238, AY551237, AY551236, AY551235, AY551234, AY551233, AY551232, AY551231, AY551230, AY551229, AY551228, AY551227, AY551226, AY551225, AY551224, AY551223, AY551222, AY551221, AY551220, AY551219, AY551218, AY551217, AY551216, AY551215, AY551214, AY551213, AY551212, AY551211, AY551210, AY551209, AY551208, AY551207, AY551206, AY551205, AY551204, AY551203, AY551202, AY551201, AY551200, AY551199, AY551198, AY551197, AY551196, AY551195, AY551194, AY551193, AY551192, AY551191, AY551190, AY551189, AY551188, AY501434, AY501433, AY501432, AY501431, AY498859, AY376459, AY376458 AY884233, AY884232, AY884231, AY884230, AY884229, AY884228, AY884227, AY884226, AY884225, AY884224, AY884223, AY884222, AY884221, AY884220, AY884219, AY884218, AY884217, AY884216, AY728475, AY728474, AY728473, AY728472, AY728471, AY728470, AY728469, AY728468, AY728467, AY728466, AY728465, AY728464, AY728463, AY728462, AY728461, AY728460, AY728459, AY728458, AY728457, AY728456, AY728455, AY728454, AY728453, AY728452, AY728451, AY728450, AY728449, AY728448, AY728447, AY728446, AY728445, AY728444, AY728443, AY728442, AY728441, AY728440, AY728439, AY728438, AY728437, AY728436, AY728435 AY728434, AY728433, AY728432, AY728431, AY728430, AY728429, AY728428, AY728427, AY728426, AY728425, AY728424, AY728423, AY728422, AY728421, AY728420, AY728419, AY728418, AY728417, AY728416, AY728415, AY728414, AY728413, AY728412, AY728411, AY728410, AY728409, AY728408, AY728407, AY728406, AY728405, AY728404, AY728403, AY728402, AY728401, AY728400, AY728399, AY728398, AY728397, AY728396, AY728395, AY728394, AY728393, AY728392, AY728391, AY728390, AY728389, AY728388, AY851149, AY851148, AY851147, AY851146, AY851145, AY851144 or AY599420.

In some embodiments, the miRNA is selected from one of the sequences disclosed in U.S. published patent application No. 2005/0144669 A1 or U.S. published patent application No. 2006/0130176 A1, each incorporated herein by reference.

In some embodiments, modifications can be included in the amiRNA precursor of the present invention so long as the modification does not affect the basic secondary structure of the synthetic amiRNA precursor produced in the cell so that the amiRNA can be properly formed and processed by the cell. As used herein, the term “modification” includes substitution and/or any change from the starting or natural miRNA precursor. With the restriction noted above in mind any number and combination of modifications can be incorporated into the amiRNA precursor. Possible modification include but are not limited to the following: (1) replacement of one or more potential GU base pairs in the predicted secondary structure of the amiRNA precursor with one or more CG or AT base pairs; (2) replacement of one or more potential GC base pairs in the predicted secondary structure of the amiRNA precursor with one or more AT base pairs; (3) replacement of one or more potential AT base pairs in the predicted secondary structure of the amiRNA precursor with one or more CG base pairs; (4) removing some parts of native pre-miRNAs; and (5) adding some designed RNA to native pre-miRNAs. Where multiple modifications are present, they may be the same or different. Modifications to bases, sugar moieties, the phosphate backbone, and their combinations are contemplated.

Examples of modifications contemplated for the phosphate backbone include phosphonates, including methylphosphonate, phosphorothioate, and phosphotriester modifications such as alkylphosphotriesters, and the like. Examples of modifications contemplated for the sugar moiety include 2′-alkyl pyrimidine, such as 2′-O-methyl, 2′-fluoro, amino, and deoxy modifications and the like (see, e.g., Amarzguioui et al. (2003) Nucleic Acids Research 31:589-595). Examples of modifications contemplated for the base groups include abasic sugars, 2-O-alkyl modified pyrimidines, 4-thiouracil, 5-bromouracil, 5-iodouracil, and 5-(3-aminoallyl)-uracil and the like. Locked nucleic acids, or LNA's, could also be incorporated. Many other modifications are known to the skilled artisan and can be used so long as the above criteria are satisfied. Examples of modifications are also disclosed in U.S. Pat. Nos. 5,684,143, 5,858,988 and 6,291,438 and in U.S. published patent application Nos. 2004/0203145 A1, 2006/0252722 A1 and 2007/0032441 A1, each incorporated herein by reference. Other modifications are disclosed in Herdewijn (2000) Antisense Nucleic Acid Drug Dev 10:297-310; Eckstein (2000) Antisense Nucleic Acid Drug Dev 10:117-21; Rusckowski et al. (2000) Antisense Nucleic Acid Drug Dev 10:333-345; Stein et al. (2001) Antisense Nucleic Acid Drug Dev 11:317-25; and Vorobjev et al. (2001) Antisense Nucleic Acid Drug Dev 11:77-85, each incorporated herein by reference.

In some embodiments, the above miRNAs, as well as those disclosed herein, have been modified to be directed to a specific target as described herein.

In some embodiments the target RNA is an RNA of a plant pathogen, such as a plant virus or plant viroid. In some embodiments, the amiRNA directed against the plant pathogen RNA is operably linked to a pathogen-inducible promoter. In some embodiments, the target RNA is an mRNA. The target sequence in an mRNA may be a non-coding sequence (such as an intron sequence, 5′ untranslated region and 3′ untranslated region), a coding sequence or a sequence involved in mRNA splicing. Targeting the amiRNA to an intron sequence compromises the maturation of the mRNA. Targeting the amiRNA to a sequence involved in mRNA splicing influences the maturation of alternative splice forms providing different protein isoforms.

In some embodiments there are provided cells, plants, and seeds comprising the polynucleotides of the invention, and/or produced by the methods of the invention. In some embodiments, the cells, plants, and/or seeds comprise a nucleic acid construct comprising a modified plant miRNA precursor, as described herein. In some embodiments, the modified plant miRNA precursor in the nucleic acid construct is operably linked to a promoter. The promoter initiates and mediates transcription of the antisense strand of the amiRNA precursor, i.e., initiates and mediates transcription of the synthetic amiRNA precursor. The promoter may be any well known promoter, including constitutive promoters, inducible promoters, derepressible promoters, and the like, such as described below. The cells include prokaryotic and eukaryotic cells, including but not limited to bacteria, yeast, fungi, viral, invertebrate, vertebrate, and plant cells. Plants, plant cells, and seeds of the invention include gynosperms, monocots and dicots, including but not limited to, corn (maize), rice, wheat, oats, barley, millet, sorghum, soy, sunflower, safflower, canola, alfalfa, cotton, Arabidopsis, and tobacco.

In another embodiment, there is provided a method for down regulating multiple target RNAs comprising introducing into a cell a nucleic acid construct encoding a multiple number of amiRNAs. One amiRNA in the multiple amiRNAs is complementary to a region of one of the target RNAs. In some embodiments, an amiRNA is fully complementary to the region of the target RNA. In some embodiments, an amiRNA is complementary and includes the use of G-U base pairing, i.e. the GU wobble, to otherwise be fully complementary. In some embodiments, the first ten nucleotides of the amiRNA (counting from the 5′ end of the amiRNA) are fully complementary to a region of the target RNA and the remaining nucleotides may include mismatches and/or bulges with the target RNA. In some embodiments an amiRNA comprises about 10-200 nucleotides, about 10-15, 15-20, 19, 20, 21, 22, 23, 24, 25, 26, 27, 25-30, 30-50, 50-100, 100-150, or about 150-200 nucleotides. The binding of an amiRNA to its complementary sequence in the target RNA results in cleavage of the target RNA. In some embodiments, the binding of the amiRNA to the complementary sequence in the target RNA results in translational repression. In some embodiments, the amiRNA is a miRNA that has been modified such that the amiRNA is fully complementary to the target sequence of the target RNA. In some embodiments, the amiRNA is a native plant miRNA that has been modified such that the amiRNA is fully complementary to the target sequence of the target RNA. In some embodiments, the amiRNA is operably linked to a promoter. The promoter initiates and mediates transcription of the antisense strand of the amiRNA precursor, i.e., initiates and mediates transcription of the synthetic amiRNA precursor. In some embodiments, the multiple amiRNAs are linked one to another so as to form a single transcript when expressed. In some embodiments, the nucleic acid construct comprises a promoter operably linked to the amiRNA.

In some embodiments, the nucleic acid construct encodes amiRNAs for suppressing a multiple number of target sequences. The nucleic acid construct encodes at least two amiRNAs. In some embodiments, each amiRNA is substantially complementary to a target or which is modified to be complementary to a target as described herein. In some embodiments, the nucleic acid construct encodes for 2-30 or more amiRNAs, for example 3-40 or more amiRNAs, for example 3-45 or more amiRNAs, and for further example, multimers of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or more amiRNAs. In some embodiments, the multiple amiRNAs are linked one to another so as to form a single transcript when expressed.

In some embodiments, polymeric miRNA precursors that are amiRNA precursors consisting of more than one amiRNA precursor units are provided. The polymeric amiRNA precurosors can either be hetero-polymeric with different amiRNA precursors, or homo-polymeric containing several units of the same amiRNA precursor. Hetero-polymeric miRNA precursors are able to produce different mature amiRNAs. See for example, U.S published patent application No. 2006/0130176, incorporated herein by reference. Homo-polymeric miRNA precursors are able to produce different mature amiRNAs. See for example, U.S published patent application No. 2006/0130176, incorporated herein by reference. In a similar manner, hetero- or homo-polymeric amiRNA precursors are produced that contain any number of monomer units.

In some embodiments, the nucleic acid construct comprises multiple polynucleotides, each polynucleotide encoding a separate amiRNA precursor. The polynucleotides are operably linked one to another such that they may be placed under the control of a single promoter. The promoter initiates and mediates transcription of the antisense strand of the amiRNA precursor, i.e., initiates and mediates transcription of the synthetic amiRNA precursor. In some embodiments, the multiple polynucleotides are linked one to another so as to form a single transcript containing the multiple amiRNA precursors when expressed. The single transcript is processed in the host cells to produce multiple mature amiRNAs, each capable of downregulating its target gene. As many polynucleotides encoding the amiRNA precursors as desired can be linked together, with the only limitation being the ultimate size of the transcript. It is well known that transcripts of 8-10 kb can be produced in plants. Thus, it is possible to form a nucleic acid construct comprising multimeric polynucleotides encoding 2-30 or more amiRNA precursors, for example 3-40 or more amiRNA precursors, for example 3-45 or more amiRNA precursors, and for further example, multimers of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or more amiRNA precursors.

In some embodiments, the nucleic acid construct further comprises a promoter operably linked to the polynucleotide encoding the multiple number of amiRNAs. The promoter initiates and mediates transcription of the antisense strand of the amiRNA precursor, i.e., initiates and mediates transcription of the synthetic amiRNA precursor. In some embodiments, the nucleic acid construct lacking a promoter is designed and introduced in such a way that it becomes operably linked to a promoter upon integration in the host genome. In some embodiments, the nucleic acid construct is integrated using recombination, including site-specific recombination. See, for example, PCT International published application No. WO 99/25821, herein incorporated by reference. In some embodiments, the nucleic acid construct is an RNA. In some embodiments, the nucleic acid construct comprises at least one recombination site, including site-specific recombination sites. In some embodiments the nucleic acid construct comprises at least one recombination site in order to facilitate integration, modification, or cloning of the construct. In some embodiments the nucleic acid construct comprises two site-specific recombination sites flanking the amiRNA precursor. In some embodiments the site-specific recombination sites include FRT sites, lox sites, or att sites, including attB, attL, attP or attR sites. See, for example, PCT International published application No. WO 99/25821, and U.S. Pat. Nos. 5,888,732, 6,143,557, 6,171,861, 6,270,969, and 6,277,608, herein incorporated by reference.

In some embodiments, the amiRNA precursor is inserted into an intron in a gene or a transgene of a cell or plant. If the gene has multiple introns, amiRNA precursors, which can be the same or different, can be inserted into each intron. In some embodiments the amiRNA precursor inserted into an intron is a polymeric amiRNA precursor. During RNA splicing, introns are released from primary RNA transcripts and therefore, can serve as precursors for amiRNAs. Most introns contain a splicing donor site at the 5′ end, splicing acceptor site at the 3′ end and a branch site within the intron. The branch site is important for intron maturation—without it, an intron can not be excised and released from the primary RNA transcript. A branch site is usually located 20-50 nt upstream of the splicing acceptor site, whereas distances between the splice donor site and the branch site are largely variable among different introns. Thus, in some embodiments, the amiRNA precursor is inserted into an intron between the splicing donor site and the branch site.

In some embodiments the target RNA is an RNA of a plant pathogen, such as a plant virus or plant viroid. In some embodiments, the amiRNA directed against the plant pathogen RNA is operably linked to a pathogen-inducible promoter. In some embodiments, the target RNA is an mRNA. The target sequence in an mRNA may be an intron sequence, a coding sequence or a sequence involved in mRNA splicing. Targeting the amiRNA to an intron sequence compromises the maturation of the mRNA. Targeting the amiRNA to a sequence involved in mRNA splicing influences the maturation of alternative splice forms providing different protein isoforms. In some embodiments, the target includes genes affecting agronomic traits, insect resistance, disease resistance, herbicide resistance, sterility, grain characteristics, and commercial products.

In some embodiments there are provided cells, plants, and seeds comprising the nucleic acid construct encoding multiple amiRNAs of the invention, and/or produced by the methods of the invention. In some embodiments, the cells, plants, and/or seeds comprise a nucleic acid construct comprising multiple polynucleotides, each encoding a plant amiRNA precursor, as described herein. In some embodiments, the multiple polynucleotides are operably linked to a promoter. The promoter initiates and mediates transcription of the antisense strand of the amiRNA precursor, i.e., initiates and mediates transcription of the synthetic amiRNA precursor. The promoter may be any well known promoter, including constitutive promoters, inducible promoters, derepressible promoters, and the like, such as described below. The polynucleotides encoding the amiRNA precursors are linked together. In some embodiments, the multiple polynucleotides are linked one to another so as to form a single transcript containing the multiple amiRNA precursors when expressed in the cells, plants or seeds. The cells include prokaryotic and eukaryotic cells, including but not limited to bacteria, yeast, fungi, viral, invertebrate, vertebrate, and plant cells. Plants, plant cells, and seeds of the invention include gynosperms, monocots and dicots, including but not limited to, corn (maize), rice, wheat, oats, barley, millet, sorghum, soy, sunflower, safflower, canola, alfalfa, cotton, Arabidopsis, and tobacco.

In some embodiments, there are provided vectors that can be used to express pre-amiRNAs or pre-miRNAs in both the sense and antisense direction. The vectors are useful for screening candidate sequences for new synthetic amiRNA precursors. The vectors are also useful for comparing expression levels of different synthetic amiRNA precursors with different folding structures by agroinfiltration. In one embodiment, the vector comprises a T-DNA binary vector with two cloning sites. In one embodiment, any suitable T-DNA binary vector can be used to produce the vectors for screening and comparing amiRNA precursors. A first cloning site is downstream of a first plant operable promoter. In one embodiment, the first cloning site is a multiple restriction site. A second cloning site is downstream of a second plant operable promoter. In one embodiment the second cloning site is a Gateway cassette (Hartley et al. (2000) Genome Res 10:1788-1795; Earley et al. (2006) The Plant J 45:616-629). In some embodiments, any suitable plant operable promoter, including those disclosed herein, can be used as a promoter in the vectors according to the present invention. In one embodiment, the first promoter is a CaMV 35S promoter. In another embodiment, the second promoter is a synthetic G10-90 promoter.

The present invention concerns methods and compositions useful in suppression of a target sequence and/or validation of function. The invention also relates to a method for using microRNA (miRNA) mediated RNA interference (RNAi) to silence or suppress a target sequence to evaluate function, or to validate a target sequence for phenotypic effect and/or trait development. Specifically, the invention relates to constructs comprising small nucleic acid molecules, amiRNAs, capable of inducing silencing, and methods of using these amiRNAs to selectively silence target sequences.

RNA interference refers to the process of sequence-specific post-transcriptional gene silencing in animals mediated by short interfering RNAs (siRNAs) (Fire et al. (1998) Nature 391:806-810). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al. (1999) Trends Genet. 15:358-363). Such protection from foreign gene expression may have evolved in response to the production of double-stranded RNAs (dsRNAs) derived from viral infection or from the random integration of transposon elements into a host genome via a cellular response that specifically destroys homologous single-stranded RNA of viral genomic RNA. The presence of dsRNA in cells triggers the RNAi response through a mechanism that has yet to be fully characterized.

The presence of long dsRNAs in cells stimulates the activity of a ribonuclease III enzyme referred to as dicer. Dicer is involved in the processing of the dsRNA into short pieces of dsRNA known as short interfering RNAs (siRNAs) (Bernstein et al. (2001) Nature 409:363-366). Short interfering RNAs derived from dicer activity are typically about 21 to about 23 nucleotides in length and comprise about 19 base pair duplexes (Elbashir et al. (2001) Genes Dev 15:188-200). Dicer has also been implicated in the excision of 21- and 22-nucleotide small temporal RNAs (stRNAs) from precursor RNA of conserved structure that are implicated in translational control (Hutvagner et al. (2001) Science 293:834-838). The RNAi response also features an endonuclease complex, commonly referred to as an RNA-induced silencing complex (RISC), which mediates cleavage of single-stranded RNA having sequence complementarity to the antisense strand of the siRNA duplex. Cleavage of the target RNA takes place in the middle of the region complementary to the antisense strand of the siRNA duplex (Elbashir et al. (2001) Genes Dev 15:188-200). In addition, RNA interference can also involve small RNA (e.g., microRNA, or miRNA) mediated gene silencing, presumably through cellular mechanisms that regulate chromatin structure and thereby prevent transcription of target gene sequences (see, e.g., Allshire, Science 297:1818-1819 2002; Volpe et al. (2002) Science 297:1833-1837; Jenuwein (2002) Science 297:2215-2218; Hall et al. (2002) Science 297:2232-2237). As such, miRNA molecules of the invention can be used to mediate gene silencing via interaction with RNA transcripts or alternately by interaction with particular gene sequences, wherein such interaction results in gene silencing either at the transcriptional or post-transcriptional level.

RNAi has been studied in a variety of systems. Fire et al. ((1998) Nature 391:806-811) were the first to observe RNAi in C. elegans. Wianny and Goetz ((1999) Nature Cell Biol 2:70) describe RNAi mediated by dsRNA in mouse embryos. Hammond et al. ((2000) Nature 404:293-296) describe RNAi in Drosophila cells transfected with dsRNA. Elbashir et al. ((2001) Nature 411:494-498) describe RNAi induced by introduction of duplexes of synthetic 21-nucleotide RNAs in cultured mammalian cells including human embryonic kidney and HeLa cells.

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

It is thought that sequence complementarity between small RNAs and their RNA targets helps to determine which mechanism, RNA cleavage or translational inhibition, is employed. It is believed that siRNAs, which are perfectly complementary with their targets, work by RNA cleavage. Some miRNAs have perfect or near-perfect complementarity with their targets, and RNA cleavage has been demonstrated for at least a few of these miRNAs. Other miRNAs have several mismatches with their targets, and apparently inhibit their targets at the translational level. Again, without being held to a particular theory on the mechanism of action, a general rule is emerging that perfect or near-perfect complementarity favors RNA cleavage, especially within the first ten nucleotides (counting from the 5′end of the miRNA), whereas translational inhibition is favored when the miRNA/target duplex contains many mismatches. The apparent exception to this is microRNA 172 (miR172) in plants. One of the targets of miR172 is APETALA2 (AP2), and although miR172 shares near-perfect complementarity with AP2 it appears to cause translational inhibition of AP2 rather than RNA cleavage.

MicroRNAs (miRNAs) are noncoding RNAs of about 19 to about 24 nucleotides (nt) in length that have been identified in both animals and plants (Lagos-Quintana et al. (2001) Science 294:853-858, Lagos-Quintana et al. (2002) Curr Biol 12:735-739; Lau et al. (2002) Science 294:858-862; Lee and Ambros (2001) Science 294:862-864; Llave et al. (2002) Plant Cell 14:1605-1619; Mourelatos et al. (2002) Genes Dev 16:720-728; Park et al. (2002) Curr Biol 12:1484-1495; Reinhart et al. (2002) Genes Dev 16:1616-1626). They are processed from longer precursor transcripts that range in size from approximately 70 to 200 nt, and these precursor transcripts have the ability to form stable hairpin structures. In animals, the enzyme involved in processing miRNA precursors is called Dicer, an RNAse III-like protein (Grishok et al. (2001) Cell 106:23-34; Hutvagner et al. (2001) Science 293:834-838; Ketting et al. (2001) Genes Dev 15:2654-2659). Plants also have a Dicer-like enzyme, DCL1 (previously named CARPEL FACTORY/SHORT INTEGUMENTS1/SUSPENSOR1), and recent evidence indicates that it, like Dicer, is involved in processing the hairpin precursors to generate mature miRNAs (Park et al. (2002) Curr Biol 12:1484-1495; Reinhart et al. (2002) Genes Dev 16:1616-1626). Furthermore, it is becoming clear from recent work that at least some miRNA hairpin precursors originate as longer polyadenylated transcripts, and several different miRNAs and associated hairpins can be present in a single transcript (Lagos-Quintana et al. (2001) Science 294:853-858; Lee et al. (2002) EMBO J. 21:4663-4670). Recent work has also examined the selection of the miRNA strand from the dsRNA product arising from processing of the hairpin by DICER (Schwartz et al. (2003) Cell 115:199-208). It appears that the stability (i.e. G:C vs. A:U content, and/or mismatches) of the two ends of the processed dsRNA affects the strand selection, with the low stability end being easier to unwind by a helicase activity. The 5′ end strand at the low stability end is incorporated into the RISC complex, while the other strand is degraded.

In animals, there is direct evidence indicating a role for specific miRNAs in development. The lin-4 and let-7 miRNAs in C. elegans have been found to control temporal development, based on the phenotypes generated when the genes producing the lin-4 and let-7 miRNAs are mutated (Lee et al. (1993) Cell 75:843-854; Reinhart et al. (2000) Nature 403-901-906). In addition, both miRNAs display a temporal expression pattern consistent with their roles in developmental timing. Other animal miRNAs display developmentally regulated patterns of expression, both temporal and tissue-specific (Lagos-Quintana et al. (2001) Science 294:853-853, Lagos-Quintana et al. (2002) Curr Biol 12:735-739; Lau et al. (2001) Science 294:858-862; Lee and Ambros (2001) Science 294:862-864), leading to the hypothesis that miRNAs may, in many cases, be involved in the regulation of important developmental processes. Likewise, in plants, the differential expression patterns of many miRNAs suggests a role in development (Llave et al. (2002) Plant Cell 14:1605-1619; Park et al. (2002) Curr Biol 12:1484-1495; Reinhart et al. (2002) Genes Dev 16:1616-1626), which has now been shown (e.g., Guo et al. (2005) Plant Cell 17:1376-1386).

MicroRNAs appear to regulate target genes by binding to complementary sequences located in the transcripts produced by these genes. In the case of lin-4 and let-7, the target sites are located in the 3′ UTRs of the target mRNAs (Lee et al. (1993) Cell 75:843-854; Wightman et al. (1993) Cell 75:855-862; Reinhart et al. (2000) Nature 403:901-906; Slack et al. (2000) Mol Cell 5:659-669), and there are several mismatches between the lin-4 and let-7 miRNAs and their target sites. Binding of the lin-4 or let-7 miRNA appears to cause downregulation of steady-state levels of the protein encoded by the target mRNA without affecting the transcript itself (Olsen and Ambros (1999) Dev Biol 216:671-680). On the other hand, recent evidence suggests that miRNAs can, in some cases, cause specific RNA cleavage of the target transcript within the target site, and this cleavage step appears to require 100% complementarity between the miRNA and the target transcript (Hutvagner and Zamore (2002) Science 297:2056-2060; Llave et al. (2002) Plant Cell 14:1605-1619), especially within the first ten nucleotides (counting from the 5′ end of the miRNA). It seems likely that miRNAs can enter at least two pathways of target gene regulation. Protein downregulation when target complementarity is <100%, and RNA cleavage when target complementarity is 100%. MicroRNAs entering the RNA cleavage pathway are analogous to the 21-25 nt short interfering RNAs (siRNAs) generated during RNA interference (RNAi) in animals and posttranscriptional gene silencing (PTGS) in plants (Hamilton and Baulcombe (1999) Science 286:950-952; Hammond et al., (2000) Nature 404:293-296; Zamore et al., (2000) Cell 31:25-33; Elbashir et al., (2001) Nature 411:494-498), and likely are incorporated into an RNA-induced silencing complex (RISC) that is similar or identical to that seen for RNAi.

Identifying the targets of miRNAs with bioinformatics has not been successful in animals, and this is probably due to the fact that animal miRNAs have a low degree of complementarity with their targets. On the other hand, bioinformatic approaches have been successfully used to predict targets for plant miRNAs (Llave et al. (2002) Plant Cell 14:1605-1619; Park et al. (2002) Curr Biol 12:1484-1495; Rhoades et al. (2002) Cell 110:513-520), and thus it appears that plant miRNAs have higher overall complementarity with their putative targets than do animal miRNAs. Most of these predicted target transcripts of plant miRNAs encode members of transcription factor families implicated in plant developmental patterning or cell differentiation. Nonetheless, biological function has not been directly demonstrated for any plant miRNA. Although Llave et al. ((2002) Science 297:2053-2056) have shown that a transcript for a SCARECROW-like transcription factor is a target of the Arabidopsis miRNA mir171, these studies were performed in a heterologous species and no plant phenotype associated with mir171 was reported.

The methods provided can be practiced in any organism in which a method of transformation is available, and for which there is at least some sequence information for the target sequence, or for a region flanking the target sequence of interest. It is also understood that two or more sequences could be targeted by sequential transformation, co-transformation with more than one targeting vector, or the construction of a DNA construct comprising more than one amiRNA sequence. The methods of the invention may also be implemented by a combinatorial nucleic acid library construction in order to generate a library of amiRNAs directed to random target sequences. The library of miRNAs could be used for high-throughput screening for gene function validation.

General categories of sequences of interest include, for example, those genes involved in regulation or information, such as zinc fingers, transcription factors, homeotic genes, or cell cycle and cell death modulators, those involved in communication, such as kinases, and those involved in housekeeping, such as heat shock proteins.

Target sequences further include coding regions and non-coding regions such as promoters, enhancers, terminators, introns and the like, which may be modified in order to alter the expression of a gene of interest. For example, an intron sequence can be added to the 5′ region to increase the amount of mature message that accumulates (see for example Buchman and Berg (1988) Mol Cell Biol 8:4395-4405); and Callis et al. (1987) Genes Dev 1:1183-1200).

The target sequence may be an endogenous sequence, or may be an introduced heterologous sequence, or transgene. For example, the methods may be used to alter the regulation or expression of a transgene, or to remove a transgene or other introduced sequence such as an introduced site-specific recombination site. The target sequence may also be a sequence from a pathogen, for example, the target sequence may be from a plant pathogen such as a virus, a mold or fungus, an insect, or a nematode. An amiRNA can be expressed in a plant which, upon infection or infestation, would target the pathogen and confer some degree of resistance to the plant. The Examples herein demonstrate the techniques to design amiRNAs to confer virus resistance/tolerance to plants. In some embodiments, two or more amiRNA sequences directed against different sequences of the virus can be used to prevent the target virus from mutating and thus evading the resistance mechanism. In some embodiments, sequences of amiRNAs can be selected so that they target a critical region of the viral RNA (e.g. active site of a silencing gene suppressor). In this case, mutation of the virus in this selected region may render the encoded protein inactive, thus preventing mutation of the virus as a way to escape the resistance mechanism. In some embodiments, an amiRNA directed towards a conserved sequence of a family of viruses would confer resistance to members of the entire family. In some embodiments, an amiRNA directed towards a sequence conserved amongst members of would confer resistance to members of the different viral families.

In plants, other categories of target sequences include genes affecting agronomic traits, insect resistance, disease resistance, herbicide resistance, sterility, grain characteristics, and commercial products. Genes of interest also include those involved in oil, starch, carbohydrate, or nutrient metabolism as well as those affecting, for example, kernel size, sucrose loading, and the like. The quality of grain is reflected in traits such as levels and types of oils, saturated and unsaturated, quality and quantity of essential amino acids, and levels of cellulose. For example, genes of the phytic acid biosynthetic pathway could be suppressed to generate a high available phosphorous phenotype. See, for example, phytic acid biosynthetic enzymes including inositol polyphosphate kinase-2 polynucleotides, disclosed in WO 02/059324, inositol 1,3,4-trisphosphate 5/6-kinase polynucleotides, disclosed in WO 03/027243, and myo-inositol 1-phosphate synthase and other phytate biosynthetic polynucleotides, disclosed in WO 99/05298, all of which are herein incorporated by reference. Genes in the lignification pathway could be suppressed to enhance digestibility or energy availability. Genes affecting cell cycle or cell death could be suppressed to affect growth or stress response. Genes affecting DNA repair and/or recombination could be suppressed to increase genetic variability. Genes affecting flowering time could be suppressed, as well as genes affecting fertility. Any target sequence could be suppressed in order to evaluate or confirm its role in a particular trait or phenotype, or to dissect a molecular, regulatory, biochemical, or proteomic pathway or network.

A number of promoters can be used, these promoters can be selected based on the desired outcome. It is recognized that different applications will be enhanced by the use of different promoters in plant expression cassettes to modulate the timing, location and/or level of expression of the amiRNA. Such plant expression cassettes may also contain, if desired, a promoter regulatory region (e.g., one conferring inducible, constitutive, environmentally- or developmentally-regulated, or cell- or tissue-specific/selective expression), a transcription initiation start site, a ribosome binding site, an RNA processing signal, a transcription termination site, and/or a polyadenylation signal.

Constitutive, tissue-preferred or inducible promoters can be employed. Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter, the SAM Synthetase promoter, the Nos promoter, the pemu promoter, and other transcription initiation regions from various plant genes known to those of skill. Examples of tissue-preferred or inducible promoters include the cinnamyl alcohol dehydrogenase promoter (U.S. Pat. No. 5,633,439), the rubisco small subunit promoter, and the GRP1-8 promoter. If low level expression is desired, weak promoter(s) may be used. Weak constitutive promoters include, for example, the core promoter of the Rsyn7 promoter (WO 99/43838 and U.S. Pat. No. 6,072,050), the core 35S CaMV promoter, and the like. Other constitutive promoters include, for example, U.S. Pat. Nos. 5,608,149; 5,608,144; 5,604,121; 5,569,597; 5,466,785; 5,399,680; 5,268,463; and 5,608,142. See also, U.S. Pat. No. 6,177,611, herein incorporated by reference.

Examples of inducible promoters are the Adh1 promoter which is inducible by hypoxia or cold stress, the Hsp70 promoter which is inducible by heat stress, the PPDK promoter and the PEP (phophoenol pyruvate) carboxylase promoter which are both inducible by light. Also useful are promoters which are chemically inducible, such as the In2-2 promoter which is safener induced (U.S. Pat. No. 5,364,780), the ERE promoter which is estrogen induced, and the Axig1 promoter which is auxin induced and tapetum specific but also active in callus (PCT International published application No. WO 02/04699). Other examples of inducible promoters include the GVG and XVE promoters, which are induced by glucocorticoids and estrogen, respectively (U.S. Pat. No. 6,452,068).

Examples of promoters under developmental control include promoters that initiate transcription preferentially in certain tissues, such as leaves, roots, fruit, seeds, or flowers. An exemplary promoter is the anther specific promoter 5126 (U.S. Pat. Nos. 5,689,049 and 5,689,051). Examples of seed-preferred promoters include, but are not limited to, 27 kD gamma zein promoter and waxy promoter (Boronat et al. (1986) Plant Sci 47:95-102; Reina et al. (1990) Nucl Acids Res 18(21):6426; Kloesgen et al. (1986) Mol. Gen. Genet. 203:237-244). Promoters that express in the embryo, pericarp, and endosperm are disclosed in U.S. Pat. No. 6,225,529 and PCT International published application No. WO 00/12733. The disclosures of each of these are incorporated herein by reference in their entirety.

In some embodiments it will be beneficial to express the gene from an inducible promoter, particularly from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also PCT International published application No. WO 99/43819, herein incorporated by reference.

Of interest are promoters that are expressed locally at or near the site of pathogen infection. See, for example, Marineau et al. (1987) Plant Mol Biol 9:335-342; Matton et al. (1989) Molecular Plant-Microbe Interactions 2:325-331; Somsisch et al. (1986) Proc Natl Acad Sci USA 83:2427-2430; Somsisch et al. (1988) Mol Gen Genet. 2:93-98; and Yang (1996) Proc Natl Acad Sci USA 93:14972-14977. See also, Chen et al. (1996) Plant J 10:955-966; Zhang et al. (1994) Proc Natl Acad Sci USA 91:2507-2511; Warner et al. (1993) Plant J 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol Mol Plant Path 41:189-200).

Additionally, as pathogens find entry into plants through wounds or insect damage, a wound-inducible promoter may be used in the constructions of the polynucleotides. Such wound-inducible promoters include potato proteinase inhibitor (pin II) gene (Ryan (1990) Ann Rev Phytopath 28:425-449; Duan et al. (1996) Nature Biotech 14:494-498); wun1 and wun2, U.S. Pat. No. 5,428,148; win1 and win2 (Stanford et al. (1989) Mol Gen Genet. 215:200-208); systemin (McGurl et al. (1992) Science 225:1570-1573); WIP1 (Rohmeier et al. (1993) Plant Mol Biol 22:783-792; Eckelkamp et al. (1993) FEBS Lett 323:73-76); MPI gene (Corderok et al. (1994) Plant J 6(2):141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize ln2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc Natl Acad Sci USA 88:10421-10425 and McNellis et al. (1998) Plant J 14(2):247-257) and tetracycline-inducible and tetracycline-repressible promoters (see, for example, Gatz et al. (1991) Mol Gen Genet. 227:229-237, and U.S. Pat. Nos. 5,814,618 and 5,789,156), herein incorporated by reference.

Tissue-preferred promoters can be utilized to target enhanced expression of a sequence of interest within a particular plant tissue. Tissue-preferred promoters include Yamamoto et al. (1997) Plant J 12(2):255-265; Kawamata et al. (1997) Plant Cell Physiol 38(7):792-803; Hansen et al. (1997) Mol Gen Genet. 254(3):337-343; Russell et al. (1997) Transgenic Res 6(2):157-168; Rinehart et al. (1996) Plant Physiol 112(3):1331-1341; Van Camp et al. (1996) Plant Physiol 112(2):525-535; Canevascini et al. (1996) Plant Physiol 112(2):513-524; Yamamoto et al. (1994) Plant Cell Physiol 35(5):773-778; Lam (1994) Results Probl Cell Differ 20:181-196; Orozco et al. (1993) Plant Mol Biol 23(6):1129-1138; Matsuoka et al. (1993) Proc Natl Acad Sci USA 90(20):9586-9590; and Guevara-Garcia et al. (1993) Plant J 4(3):495-505. Such promoters can be modified, if necessary, for weak expression.

Leaf-preferred promoters are known in the art. See, for example, Yamamoto et al. (1997) Plant J 12(2):255-265; Kwon et al. (1994) Plant Physiol 105:357-67; Yamamoto et al. (1994) Plant Cell Physiol 35(5):773-778; Gotor et al. (1993) Plant J 3:509-18; Orozco et al. (1993) Plant Mol Biol 23(6):1129-1138; and Matsuoka et al. (1993) Proc Natl Acad Sci USA 90(20):9586-9590. In addition, the promoters of cab and RUBISCO can also be used. See, for example, Simpson et al. (1958) EMBO J. 4:2723-2729 and Timko et al. (1988) Nature 318:57-58.

Root-preferred promoters are known and can be selected from the many available from the literature or isolated de novo from various compatible species. See, for example, Hire et al. (1992) Plant Mol Biol 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol Biol 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi ((1991) Plant Science (Limerick) 79(1):69-76) describe their analysis of the promoters of the highly expressed rolC and rolD root-inducing genes of Agrobacterium rhizogenes. They concluded that enhancer and tissue-preferred DNA determinants are dissociated in those promoters. Teeri et al. ((1989) EMBO J. 8(2):343-350) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene. The TR1′ gene, fused to nptII (neomycin phosphotransferase II) showed similar characteristics. Additional root-preferred promoters include the VfENOD-GRP3 gene promoter (Kuster et al. (1995) Plant Mol Biol 29(4):759-772); and rolB promoter (Capana et al. (1994) Plant Mol Biol 25(4):681-691. See also U.S. Pat. Nos. 5,837,876; 5,750,386; 5,633,363; 5,459,252; 5,401,836; 5,110,732; and 5,023,179. The phaseolin gene (Murai et al. (1983) Science 23:476-482 and Sengopta-Gopalen et al. (1988) Proc Natl Acad Sci USA 82:3320-3324.

Transformation protocols as well as protocols for introducing nucleotide sequences into plants may vary depending on the type of plant or plant cell, i.e., monocot or dicot, targeted for transformation. Suitable methods of introducing the DNA construct include microinjection (Crossway et al. (1986) Biotechniques 4:320-334; and U.S. Pat. No. 6,300,543), sexual crossing, electroporation (Riggs et al. (1986) Proc Natl Acad Sci USA 83:5602-5606), Agrobacterium-mediated transformation (Townsend et al., U.S. Pat. No. 5,563,055; and U.S. Pat. No. 5,981,840), direct gene transfer (Paszkowski et al. (1984) EMBO J. 3:2717-2722), and ballistic particle acceleration (see, for example, U.S. Pat. No. 4,945,050; U.S. Pat. No. 5,879,918; U.S. Pat. No. 5,886,244; U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6:923-926). Also see Weissinger et al. (1988) Ann Rev Genet 22:421-477; Sanford et al. (1987) Particulate Science and Technology 5:27-37 (onion); Christou et al. (1988) Plant Physiol 87:671-674 (soybean); Finer and McMullen (1991) In Vitro Cell Dev Biol 27P:175-182 (soybean); Singh et al. (1998) Theor Appl Genet 96:319-324 (soybean); Datta et al. (1990) Biotechnology 8:736-740 (rice); Klein et al. (1988) Proc Natl Acad Sci USA 85:4305-4309 (maize); Klein et al. (1988) Biotechnology 6:559-563 (maize); U.S. Pat. No. 5,240,855; U.S. Pat. No. 5,322,783; U.S. Pat. No. 5,324,646; Klein et al. (1988) Plant Physiol 91:440-444 (maize); Fromm et al. (1990) Biotechnology 8:833-839 (maize); Hooykaas-Van Slogteren et al. (1984) Nature 311:763-764; U.S. Pat. No. 5,736,369 (cereals); Bytebier et al. (1987) Proc Natl Acad Sci USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor Appl Genet 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); and U.S. Pat. No. 5,736,369 (meristem transformation), all of which are herein incorporated by reference.

The nucleotide constructs may be introduced into plants by contacting plants with a virus or viral nucleic acids. Generally, such methods involve incorporating a nucleotide construct of the invention within a viral DNA or RNA molecule. Further, it is recognized that useful promoters encompass promoters utilized for transcription by viral RNA polymerases. Methods for introducing nucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

In some embodiments, transient expression may be desired. In those cases, standard transient transformation techniques may be used. Such methods include, but are not limited to viral transformation methods, and microinjection of DNA or RNA, as well other methods well known in the art.

The cells from the plants that have stably incorporated the nucleotide sequence may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic imparted by the nucleotide sequence of interest and/or the genetic markers contained within the target site or transfer cassette. Two or more generations may be grown to ensure that expression of the desired phenotypic characteristic is stably maintained and inherited and then seeds harvested to ensure expression of the desired phenotypic characteristic has been achieved.

Initial identification and selection of cells and/or plants comprising the DNA constructs may be facilitated by the use of marker genes. Gene targeting can be performed without selection if there is a sensitive method for identifying recombinants, for example if the targeted gene modification can be easily detected by PCR analysis, or if it results in a certain phenotype. However, in most cases, identification of gene targeting events will be facilitated by the use of markers. Useful markers include positive and negative selectable markers as well as markers that facilitate screening, such as visual markers. Selectable markers include genes carrying resistance to an antibiotic such as spectinomycin (e.g. the aada gene, Svab et al. (1990) Plant Mol Biol 14:197-205), streptomycin (e.g., aada, or SPT, Svab et al. (1990) Plant Mol Biol 14:197-205; Jones et al. (1987) Mol Gen Genet 210:86), kanamycin (e.g., nptII, Fraley et al. (1983) Proc Natl Acad Sci USA 80:4803-4807), hygromycin (e.g., HPT, Vanden Elzen et al. (1985) Plant Mol Biol 5:299), gentamycin (Hayford et al. (1988) Plant Physiol 86:1216), phleomycin, zeocin, or bleomycin (Hille et al. (1986) Plant Mol Biol 7:171), or resistance to a herbicide such as phosphinothricin (bar gene), or sulfonylurea (acetolactate synthase (ALS)) (Charest et al. (1990) Plant Cell Rep 8:643), genes that fulfill a growth requirement on an incomplete media such as HIS3, LEU2, URA3, LYS2, and TRP1 genes in yeast, and other such genes known in the art. Negative selectable markers include cytosine deaminase (codA) (Stougaard (1993) Plant J. 3:755-761), tms2 (DePicker et al. (1988) Plant Cell Rep 7:63-66), nitrate reductase (Nussame et al. (1991) Plant J 1:267-274), SU1 (O'Keefe et al. (1994) Plant Physiol. 105:473-482), aux-2 from the T1 plasmid of Agrobacterium, and thymidine kinase. Screenable markers include fluorescent proteins such as green fluorescent protein (GFP) (Chalfie et al. (1994) Science 263:802; U.S. Pat. No. 6,146,826; U.S. Pat. No. 5,491,084; and WO 97/41228), reporter enzymes such as β-glucuronidase (GUS) (Jefferson (1987) Plant Mol Biol Rep 5:387; U.S. Pat. No. 5,599,670; U.S. Pat. No. 5,432,081), β3-galactosidase (lacZ), alkaline phosphatase (AP), glutathione S-transferase (GST) and luciferase (U.S. Pat. No. 5,674,713; Ow et al. (1986) Science 234:856-859), visual markers like anthocyanins such as CRC (Ludwig et al. (1990) Science 247:449-450) R gene family (e.g. Lc, P, S), A, C, R-nj, body and/or eye color genes in Drosophila, coat color genes in mammalian systems, and others known in the art.

One or more markers may be used in order to select and screen for gene targeting events. One common strategy for gene disruption involves using a target modifying polynucleotide in which the target is disrupted by a promoterless selectable marker. Since the selectable marker lacks a promoter, random integration events are unlikely to lead to transcription of the gene. Gene targeting events will put the selectable marker under control of the promoter for the target gene. Gene targeting events are identified by selection for expression of the selectable marker. Another common strategy utilizes a positive-negative selection scheme. This scheme utilizes two selectable markers, one that confers resistance (R+) coupled with one that confers a sensitivity (S+), each with a promoter. When this polynucleotide is randomly inserted, the resulting phenotype is R+/S+. When a gene targeting event is generated, the two markers are uncoupled and the resulting phenotype is R+/S−. Examples of using positive-negative selection are found in Thykjaer et al. (1997) Plant Mol Biol 35:523-530; and PCT International published application No. WO 01/66717, which are herein incorporated by reference.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Russell, 1984, Molecular biology of plants: a laboratory course manual (Cold Spring Harbor Laboratory Press, Cold Spring Harbor, N.Y.); Anand, Techniques for the Analysis of Complex Genomes, (Academic Press, New York, 1992); Guthrie and Fink, Guide to Yeast Genetics and Molecular Biology (Academic Press, New York, 1991); Harlow and Lane, 1988, Antibodies, (Cold Spring Harbor Laboratory Press, Cold Spring Harbor, N.Y.); Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Fire et al., RNA Interference Technology: From Basic Science to Drug Development, Cambridge University Press, Cambridge, 2005; Schepers, RNA Interference in Practice, Wiley-VCH, 2005; Engelke, RNA Interference (RNAi): The Nuts & Bolts of siRNA Technology, DNA Press, 2003; Gott, RNA Interference, Editing, and Modification: Methods and Protocols (Methods in Molecular Biology), Human Press, Totowa, N.J., 2004; Sohail, Gene Silencing by RNA Interference: Technology and Application, CRC, 2004; Clarke and Sanseau, microRNA: Biology, Function & Expression (Nuts & Bolts series), DNA Press, 2006.

### EXAMPLES

The present invention is described by reference to the following Examples, which are offered by way of illustration and are not intended to limit the invention in any manner. Standard techniques well known in the art or the techniques specifically described below were utilized.

### Example 1

### Vector Construction

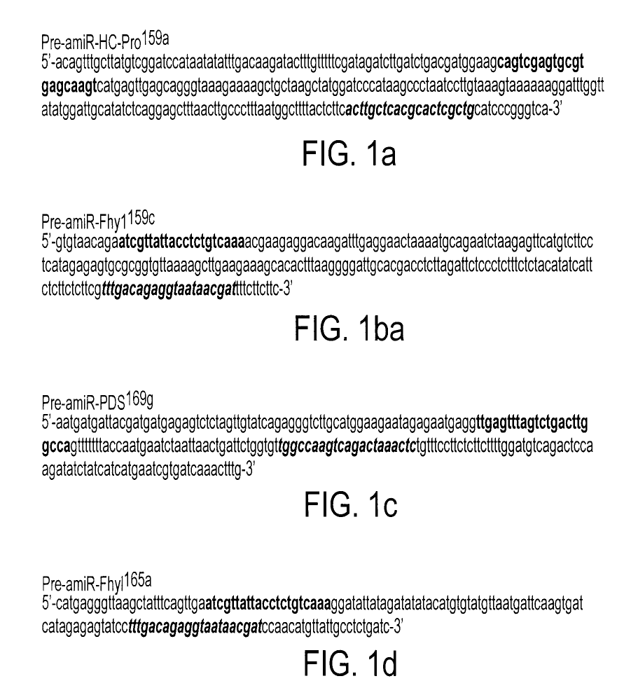

Four pre-amiRNAs, pre-amiR-HC-Pro^{159a}, pre-amiR-Fhy1^{159c}, pre-amiR-PDS^{169g} and pre-amiR-Fhy1^{165a} were constructed. Pre-amiR-HC-Pro^{159a}, was generated from pre-miR159a and pre-amiR-Fhy1^{159c} from pre-miR159c, pre-amiR-PDS^{169g} from pre-miR169g, pre-amiR-Fhy1^{165a}, from pre-miR165a. Methods for the construction of these pre-amiRNAs were described in a recent publication (Niu et al. (2006) Nat Biotechnol 24:1420-1428). Sequences of these pre-amiRNAs are shown in FIG. 1. Mature amiRNAs and their reverse complementary sequences (amiRNA* strands) are in bold italic and bold, respectively. The remaining nucleotide sequences are native pre-miRNAs backbone sequences. These four pre-amiRNAs were transferred into the Gateway system pENTR vector (Invitrogen) to obtain pENTR-pre-amiR-HC-Pro^{159a}, pENTR-pre-amiR-Fhy1^{159c}, pENTR-pre-amiR-PDS^{169g} and pENTR-pre-amiR-Fhy1^{165a.}

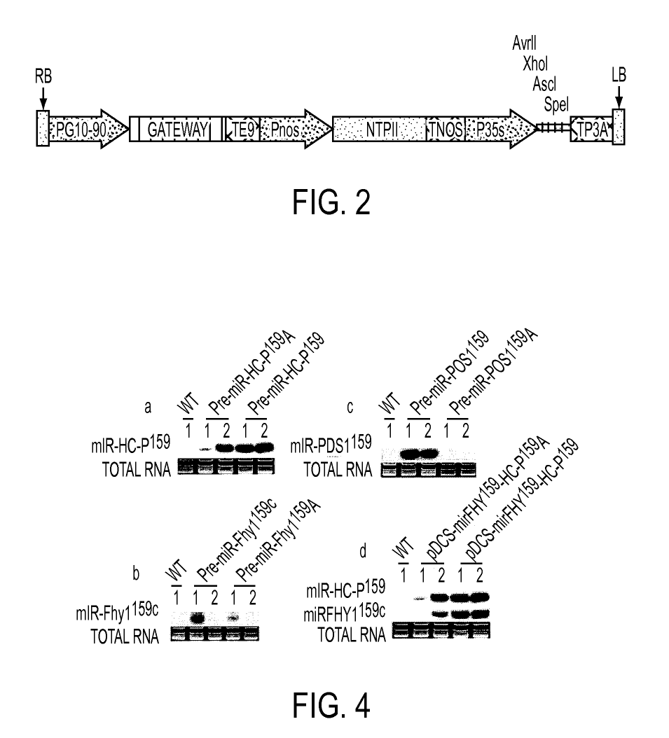

A new vector, pDCS, was constructed to conveniently express these pre-amiRNAs in both the sense and the anti-sense direction. This vector is a T-DNA transformation binary vector (FIG. 2) containing two cloning sites. One is multiple restriction enzyme site down stream of a CaMV 35S promoter. The multiple restriction enzyme sites are AvrII, XhoI, AscI and SpeI in the 5′ to 3′ direction. The other cloning site is a Gateway cassette placed downstream of the synthetic G10-−90 promoter thus making pDCS a Gateway system destination vector. The two cloning sites of this vector provides another advantage because it allows the comparison of amiRNA levels produced from the sense and antisense pre-amiRNAs using Agrobacteria mediated transient transformation system.

To insert pre-amiRNAs into pDCS in both the sense and anti-sense direction, we placed an AvrII restrict enzyme site in the 5′ end and an XhoI site in the 3′ end of these pre-amiRNAs by PCR amplification. Double digestion of these DNA fragments with AvrII and XhoI should produce pre-amiRNAs with AvrII site at the 5′ and the XhoI site and at the 3′ end. The vector pDCS was opened by double digestion with AvrII and XhoI or XhoI and SpeI to obtain pDCS_{AvrII-Xho1} and pDCS_{XhoI-SpeI}, respectively. Double digested pre-amiR-HC-Pro^{159a}, was ligated with pDCS_{AvrII-Xho1} to obtain pDCS/pre-amiR-HC-Pro^{159a}, in which a 35S promoter drives the expression of the sense pre-amiR-HC-Pro^{159a}. Because of identical ends produced by AvrII and SpeI digest, ligation of the double digested pre-amiR-HC-Pro^{159a} and pDCS_{XhoI-SpeI} will also produce pDCS/pre-amiR-HC-Pro^{159a}A (A for Antisense), in which a 35S promoter drives the expression of the anti-sense pre-amiR-HC-Pro¹⁵⁹. In this manner, we constructed pDCS/pre-amiR-Fhy1^{159c}, pDCS/pre-amiR-Fhy1^{159c}A, pDCS/pre-amiR-PDS^{169g}, pDCS/pre-amiR-PDS^{169g}A pDCS/pre-amiR-Fhy1^{165a}, and pDCS/pre-amiR-Fhy1^{165a}A. Because these 8 pDCS vectors are Gateway system destination vectors, we were able to transfer pre-amiR-Fhy1^{165a} from pENTR-pre-amiR-Fhy1^{165a} into pDCS/pre-amiR-HC-Pro^{159a} and pDCS/pre-amiR-HC-Pro^{159a}A to obtain construct pDCS/pre-amiR-Fhy1^{165a}-pre-amiR-HC-Pro^{159a} and pDCS/pre-amiR-Fhy1^{165a}-pre-amiR-HC-Pro^{159a} A, respectively, in which the reference gene pre-amiR-Fhy1^{165a}s was placed downstream of the synthetic promoter G10-90. In the same manner, we assembled the other 6 constructs, pDCS/pre-amiR-HC-Pro^{159a}-pre-amiR-Fhy1^{159c}, pDCS/pre-amiR-HC-Pro^{159a}-pre-amiR-Fhy1^{159c}A, pDCS/pre-amiR-HC-Pro^{159a}-pre-amiR-PDS^{169g}, pDCS/pre-amiR-HC-Pro^{159a}-pre-amiR-PDS^{169g}A, pDCS/pre-amiR-HC-Pro^{159a}-pre-amiR-Fhy1^{165a} and pDCS/pre-amiR-HC-Pro^{159a}-pre-amiR-Fhy1^{165a}A.

### Example 2

### Transient Expression by Agro-Infiltration of N. benthamiana

Agrobacterial cells carrying constructs, pDCS/pre-amiR-Fhy1^{165a}-pre-amiR-HC-Pro^{159a}, pDCS/pre-amiR-Fhy1^{165a}-pre-amiR-HC-Pro^{159a}A, pDCS/pre-amiR-HC-Pro^{159a}-pre-amiR-Fhy1^{159c}, pDCS/pre-amiR-HC-Pro^{159a}-pre-amiR-Fhy1^{159c}A, pDCS/pre-amiR-HC-Pro^{159a}-pre-amiR-PDS^{169g}, pDCS/pre-amiR-HC-Pro^{159a}-pre-amiR-PDS^{169g}A, pDCS/pre-amiR-HC-Pro^{159a}-pre-amiR-Fhy1^{165a} and pDCS/pre-amiR-HC-Pro^{159a}-pre-amiR-Fhy1^{165a}A, were used to infiltrate N. benthamiana leaves (Llave et al. (2000) Proc Natl Acad Sci USA 97:13401-13406; Voinnet et al. (2000) Cell 103:157-167). Two days after agroinfiltration, total RNA was extracted from the infiltrated leaves for northern analysis.

### Example 3

### Northern Blot Hybridizations

Total RNA was extracted from leaves using the Trizol reagent (Invitrogen). Ten micrograms of RNA was resolved on a 15% polyacrylamide/1× TBE (8.9 mM Tris, 8.9 mM boric acid, 20 mM EDTA)/8 M urea gel and blotted to a Hybond-N⁺ membrane (Amersham). DNA oligonucleotides with the exact reverse-complementary sequence to the amiRNAs were end-labeled with ³²P-γ-ATP and T4 polynucleotide kinase (New England Biolabs) to generate high specific radioactivity probes. Hybridization was carried out using the ULTRAHyb-Oligo solution according to the manufacturer's directions (Ambion), and signals were detected by autoradiography. In each case, the probe contained the exact antisense sequence of the expected amiRNA to be detected.

### Example 4

### Anti-Sense Pre-amiRNAs is a Kind of Synthetic Pre-miRNA

A synthetic pre-amiRNA should be a totally new RNA sequence not found in plants and differs from any native miRNA precursor. The 4 pre-amiRNAs, pre-amiR-HC-Pro^{159a}, pre-amiR-Fhy1^{159c}, pre-amiR-PDS^{169g} and pre-amiR-Fhy1^{165a}, were able to generate amiRNAs (amiR-HC-Pro^{159a}, amiR-Fhy1^{159c}, amiR-PDS^{169g} and amiR-Fhy1^{165a}) when expressed in plants. All of them contain native miRNA159a, 159c, 169g and 165a precursor backbone sequences so they are not truly synthetic miRNA precursors. Sequences of anti-sense pre-amiRNAs are different from those of the sense pre-amiRNAs and are bona fide synthetic sequences.

### Example 5

### RNA Folding Structures are Different Between Sense and Anti-Sense Sequences

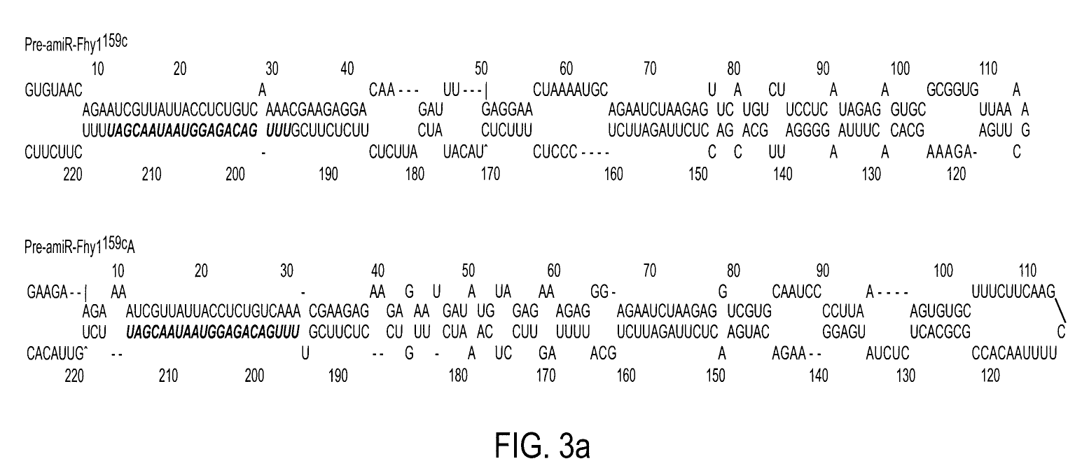

FIG. 3 shows the predicted folding structures of the 4 sense and anti-sense pre-amiRNAs. Sequences of the mature amiRNA are in bold italic. Comparing the folding structures a few interesting points emerge. First, the folding structure of the sense and anti-sense RNA appear similar. Patterns of stems and loops are similar between sense and anti-sense forms. This may explain why mature amiRNA may be generated from same site in both sense and anti-sense pre-amiRNAs. Second, both sense and anti-sense pre-amiRNAs generate the same amiRNA. For example, pre-amiR-HC-Pro^{159a} generates mature amiRNA-HC-Pro^{159a}. The sequence is 5′GUCAGCUCACGCACUCGUUCA3′ (SEQ ID NO:13). The sequence of mature miRNA generated by pre-amiR-HC-Pro^{159a}A is also 5′GUCAGCUCACGCACUCGUUCA3′ (SEQ ID NO:13). This because the amiRNA* strand is fully complementary to the amiRNA strand. Because the same amiRNA was produced by both forms of pre-amiRNAs (sense and antisense) we were able to determine the amiRNA expression levels using one RNA probe. Third, the folding structure is clearly altered in some sites. For example the end structure of pre-amiR-Fhy1^{159c} is a small loop and small stem, whereas in pre-amiR-Fhy1^{159c}A it is a big loop. This is because of disruption of G-U pairing. For RNA, G and U and are able to pair to form stem structure. In the anti-sense form, G is changed to C and U to A, and consequently loop structures are formed because C and A are unable to pair.

### Example 6

### Synthetic Pre-amiR-HC-Pro^{159a}A and Pre-amiR-Fhy1^{159c}A are able to Generate amiRNAs

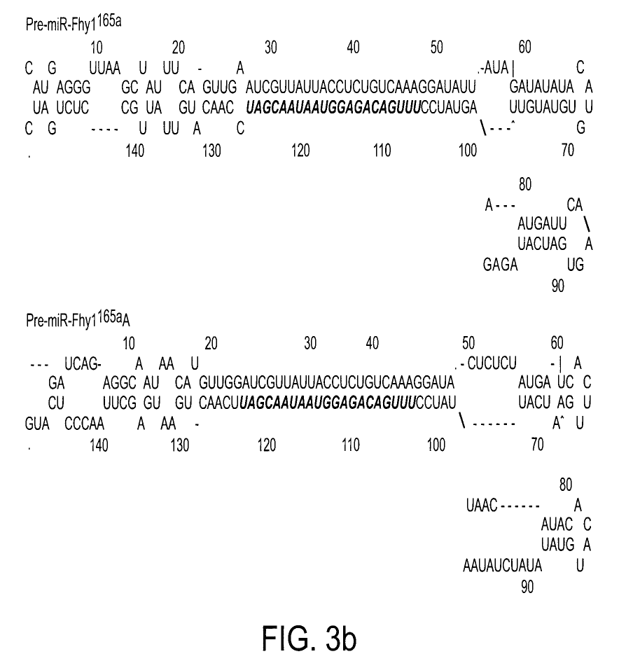

FIG. 4 shows that pre-amiR-HC-Pro^{159a}A and pre-amiR-Fhy1^{159c}A were able to generate amiRNAs (FIGS. 4a, 4b) whereas pre-amiR-PDS^{169g}A appeared unable to generate amiR-PDS^{169g} (FIG. 4c) and the same was found for pre-amiR-Fhy1^{165a}A. At present, we do not know why these two anti-sense pre-amiRNAs were unable to produce mature amiRNA. One possible reason is the alteration of RNA folding structure such that miRNAs processing proteins could not recognize these two anti-sense pre-amiRNAs. Another possible reason is that the synthetic pre-amiRNAs were processed at a different site generating a different mature amiRNA which was not detected by our probe. Because of this result, it is important to screen miRNA precursors to determine if they can be used to produce amiRNA from synthetic amiRNA precursors as described herein.

### Example 7

### Efficient Production of amiRNAs by Synthetic Pre-amiR-HC-Pro^{159a}A

Arabidopsis miRNA^{159a} precursor is one of the best miRNA precursor backbones to generate amiRNAs. Using this backbone we have efficiently generate many different amiRNAs. To test whether synthetic pre-amiR-HC-Pro^{159a}A is as efficient as pre-miR159a for the production of mature amiRNAs, we analyzed amiRNA levels produced by constructs pDCS/pre-amiR-Fhy1^{165a}-pre-amiR-HC-Pro^{159a} and pDCS/pre-amiR-Fhy1^{165a}-pre-amiR-HC-Pro^{159a}A in Nicotiana benthamiana by agro-infiltration transient expression assays. Northern blot results are shown in FIG. 4d. amiRNA signals produced by amiR-Fhy1^{165a} served as an internal control for different transient expression events (pDCS/pre-amiR-Fhy1¹⁶⁵′-pre-amiR-HC-Pro^{159a} 1, 2, pDCS/pre-amiR-Fhy1^{165a}-pre-amiR-HC-Pro^{159a}A 1, 2). Higher levels of amiR-Fhy1^{165a} indicated a higher transient expression efficiency. The level of amiR-Fhy1^{165a} in pDCS/pre-amiR-Fhy1^{165a}-pre-amiR-HC-Pro^{159a}A 2 transformations (sample A2) was lower than that in pDCS/pre-amiR-Fhy1^{165a}-pre-amiR-HC-Pro^{159a} 1 (sample 1). This indicated a reduced expression or a lower amount of pre-amiR-HC-Pro^{159a}A were tested in sample A2 than that in sample 1. AmiR-HC-Pro^{159a} levels were similar in these two transformation events indicating that the synthetic pre-amiR-HC-Pro^{159a}A was more efficient in producing amiRNAs than pre-amiR-HC-Pro^{159a}.

### Example 8

### Processing of Anti-Sense pre-miRNAs Requires DCL1

Anti-sense pre-miRNA is a type of synthetic pre-miRNA which we have previously shown to generate amiRNA when expressed in transgenic plants. To ensure that small RNAs derived from anti-sense pre-miRNA templates are indeed matured using the endogenous miRNA biogenesis pathway rather than the siRNA biogenesis pathway we investigated whether their production is dependent on DCL1. Arabidopsis DCL1 is involved in miRNAs biogenesis and required for plant normal development. Arabidopsis plants with null mutation in DCL1, such as dcll-3, are embryo lethal. Weak mutant alleles, such as dcll-9, show dramatically reduced endogenous miRNAs levels. In contrast to wild type (WT) plants, these mutant plants are smaller in stature with dark-green, narrow serrate leaves. Therefore, we investigated expression levels of amiRNAs derived from synthetic pre-miRNAs in dcll-9 mutant and WT plants.

We constructed two vectors, pBA/pre-amiR-HC-P^{159a}A and pBA/pre-amiR-P69^{159a}A, in which a 35S promoter was used to transcribe the anti-sense strand of pre-amiR-HC-P^{159a} or pre-amiR-P69^{159a}. Heterozygous +/dcll-9 were transformed by the floral dipping method using Agrobacteria carrying these vectors. Transformed seedlings were selected on selection medium and two types of transgenic seedlings were collected based on seedling morphology. One class of seedlings appeared WT in morphology (mixed genotypes +/+ or +/dcll-9) whereas the other class of seedlings had dcll-9 phenotype. Both types of seedlings grew on selection medium indicating that they were indeed transformed and therefore should express pre-amiR-HC-P^{159a}A or pre-amiR-P69^{159a}A, which are the anti-sense forms of pre-amiR-HC-P^{159a} or pre-amiR-P69^{159a}, respectively. Leaf samples were collected from 3 week-old seedlings of WT, dcll-9, and transgenic lines expressing pre-amiR-HC-P^{159a}A or pre-amiR-P69^{159a}A.

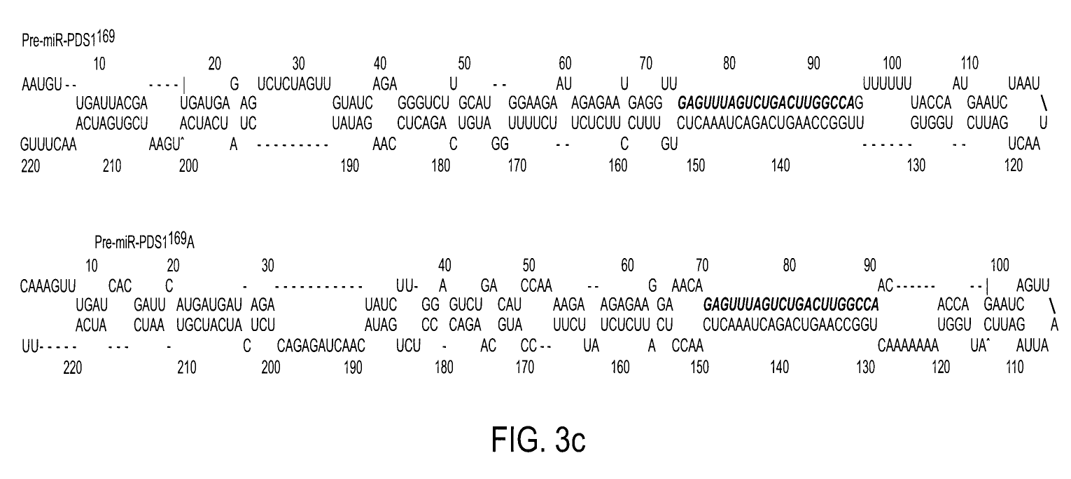

Northern blot analysis (FIG. 5) indicated that both endogenous and artificial miRNAs levels were clearly decreased in dcll-9 mutant as compared to WT plants. For example, miR-169, an endogenous miRNA, was not detectable in dcll-9 mutants and in transgenic plants showing dcll-9 phenotype (pre-miR-HC-P^{159a}A dcll-9 or pre-miR-P69^{159a}A dcll-9). On the other hand, high miR-169 expression levels were detected in WT and transgenic plants showing WT phenotype (pre-miR-HC-P^{159a}A WT or pre-miR-P69^{159a}A WT). Levels of matured artificial miRNAs (amiR-HC-P^{159a} or amiR-P69^{159a}) in dcll-9 mutant transgenic plants (pre-miR-HC-P^{159a}A dcll-9 or pre-miR-P69^{159a}A dcll-9) were considerably lower than in WT transgenic plants (pre-miR-HC-P^{159a}A wt or pre-miR-P69^{159a}A wt). These results showed that transcript from the anti-sense strand of pre-miRNAs were processed by the endogenous miRNA pathway.

### Example 9

### Expressing Anti-Sense Transcript of pre-miRNAs does not Disrupt Endogenous miRNAs Production

The anti-sense transcript of pre-amiR-HC-P^{159a} or pre-amiR-P69^{159a} is complementary to the relevant region of the native pre-miR-159a transcript except for the 21-nt sequences represented by the matured amiRNA and its star strand. It may possible that the anti-sense transcript and the native sense pre-miR-159a transcript may form dsRNAs which are then processed by the cellular siRNA pathway compromising expression of the native miR159a. As native miRNAs are important for normal plant development, disruption of miRNA biogenesis will result in plants with abnormal development, which is not acceptable for a technique used in plant engineering. To investigate this issue, we performed northern blot analysis using two-week old seedlings of transgenic Arabidopsis expressing either pre-amiR-HC-P^{159a}A or pre-amiR-P69^{159a}A. Five independent transgenic lines were analyzed for each construct.

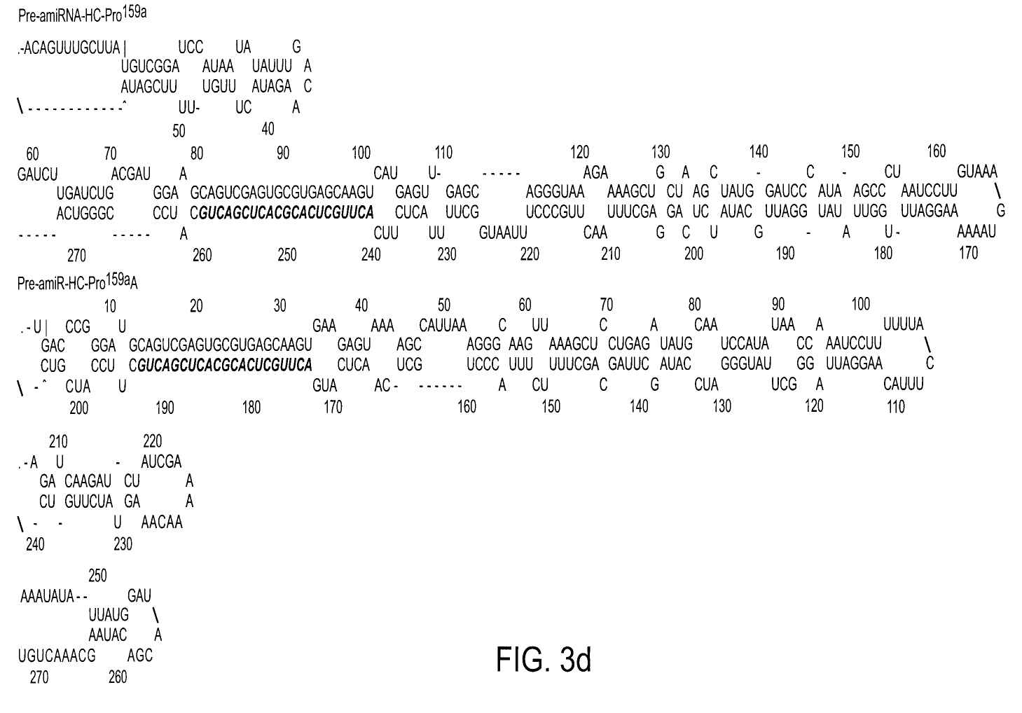

FIGS. 6a and 6b show that expression levels of amiR-HC-P^{159a} vary for lines expressing the pre-amiR-HC-P^{159a}A construct with line#4 and line#6 having a lower and a higher level, respectively. Similar variations in amiR-P69^{159a} expression levels were found amongst transgenic lines expressing pre-amiR-P69^{159a}A. As expected, no amiR-HC-P^{159a} nor amiR-P69^{159a} was detected in non-transgenic WT plants. Notwithstanding the variation in amiRNA expression levels there were no significant differences in endogenous miR-159a levels amongst WT and these different transgenic lines (FIGS. 6a and b). Our results show that expressing anti-sense transcripts of pre-miRNAs does not significantly affect endogenous miRNA production.

### Example 10

### Biological Activity of amiRNAs Expressed from Anti-Sense pre-miRNAs

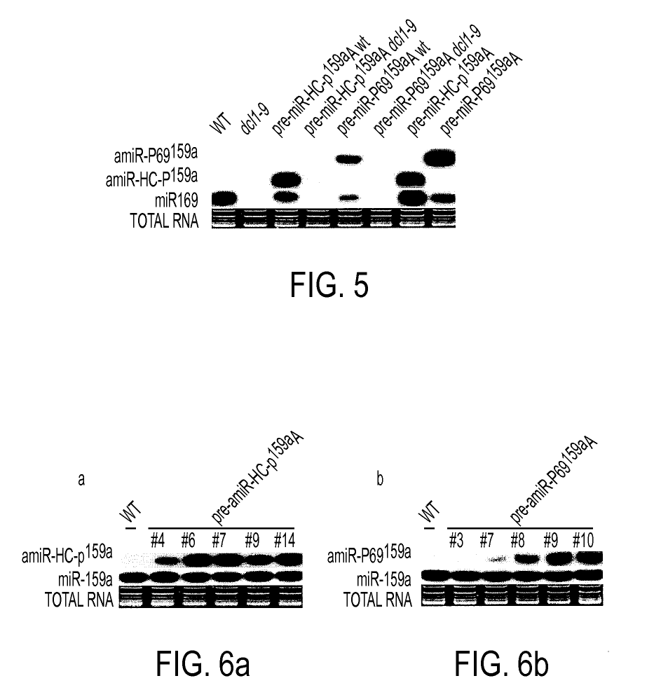

To see whether amiRNAs expressed from anti-sense pre-miRNAs have any biological activity in plants, we designed artificial miRNAs targeting the TuMV HC-Pro gene and the TYMV P69 gene, and the constructs were designed pre-amiR-HC-P^{159a}A and pre-amiR-P69^{159a}A, respectively. We generated transgenic plants expressing pre-amiR-HC-P^{159a}A and pre-amiR-P69^{159a}A from a 35S promoter. FIG. 7 shows amiRNA expression levels of independent transgenic lines. As expected, there was wide variation in the amiRNA expression level amongst transgenic lines.

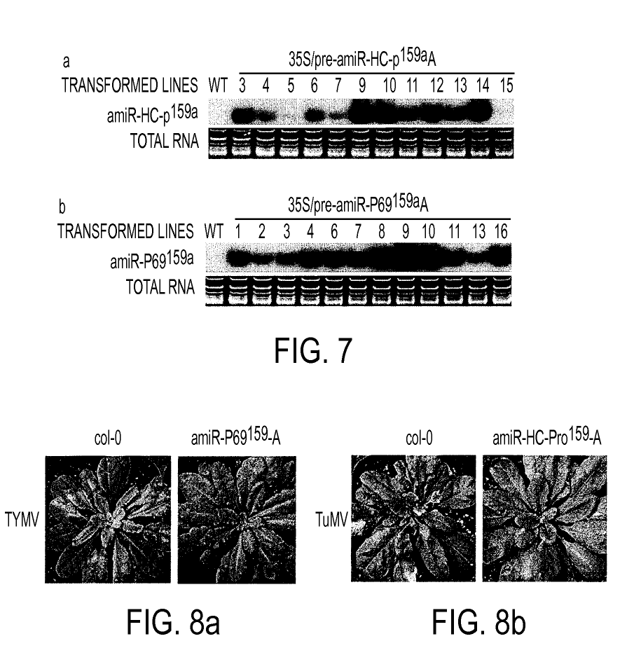

To assay for the biological function of these amiRNAs, we challenged T1 and T2 transgenic plants with the appropriate virus. Methods of TuMV and TYMV inoculation and ELISA assays for virus titters were as described by Niu et al (Niu et al. (2006) Nat Biotechnol 24:1420-1428). FIGS. 8a and 8b show that WT plants were sensitive to TuMV infection (FIG. 8b) displaying lesions on systemic leaves whereas T1 transgenic plants expressing pre-amiR-HC-P^{159a}A did not show any symptoms. These visual observations were confirmed by ELISA assay confirming that the TuMV coat protein was not detectable in new leaves of inoculated transgenic plants.

Table 1 tabulates virus resistance efficiencies of the various transgenic lines. For experiments with T1 transgenic population, each transgenic plant is derived from an independent transgenic event line. For pre-amiR-P69^{159a}A transformed plants, 30 lines out of 31 lines inoculated with TYMV showed virus resistance and the resistant efficiency was as high as 96.8%. For pre-amiR-HC-P^{159a}A transformed plants, 22 out of 26 T1 plants were resistant to TuMV. All wild type control plants inoculated succumbed to TYMV or TuMV infection.

TABLE 1
Virus Resistance Efficiencies of T1 Transgenic Lines

| | pre-amiR-P69^{159a} A | Pre-amiR-HC-p^{159a} A | Col-0 |
|---|---|---|---|
| Treatments | a/b (%) | a/b (%) | a/b (%) |
| TuMV | | 22/26 (84.6) | 0/12 (0.0) |
| TYMV | 30/31 (96.8) | | 0/12 (0.0) |
| Mock | 1/1 (100.0) | 2/2 (100.0) | 8/8 (100.0) |

Note:
a, number of resistant plants.
b, number of total inoculated plants.

Table 2 shows results obtained with T2 transgenic lines. Three lines of transgenic pre-amiR-HC-p^{159a}A plants showed 100%, 55%, and 85% virus resistance when challenged with TuMV, whereas all WT control plants and transgenic lines expressing pre-amiR-P69^{159a}A were totally sensitive to the same virus. These results demonstrated that amiR-HC-p^{159a} matured from the anti-sense transcript of pre-amiR-HC-p¹⁵⁹, were able to guide cleavage of TuMV thus rendering the expressing plants resistant to the virus. Similar experiments with transgenic lines expressing amiR-P69^{159a} generated from pre-amiR-P69^{159a}A showed that these plants were resistant to TYMV but sensitive to TuMV.

TABLE 2
Virus Resistance Efficiencies of T2 Transgenic Lines

| | TuMY | | TYMY | |
|---|---|---|---|---|
| Transformed lines | a/b | r (%) | a/b | r (%) |
| pre-amiR-HC-p^{159a} A #9 | 20/20 | 100 | 4/8 | 50 |
| pre-amiR-HC-p^{159a} A #10 | 11/20 | 55 | 1/8 | 12.5 |
| pre-amiR-HC-p^{159a} A #14 | 17/20 | 85 | 3/8 | 37.5 |
| pre-amiR-P69^{159a} A #8 | 0/11 | 0 | 19/20 | 95 |
| pre-amiR-P69^{159a} A #9 | 0/8 | 0 | 20/20 | 100 |
| pre-amiR-P69^{159a} A #10 | 0/8 | 0 | 19/20 | 95 |
| Col-0 | 0/12 | 0 | 0/12 | 0 |

Note:
a, number of resistant plants.
b, number of total inoculated plants.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A nucleic acid construct comprising a promoter operatively linked to a polynucleotide which encodes an artificial miRNA (amiRNA) precursor capable of forming a double-stranded RNA or a hairpin, wherein the amiRNA precursor comprises a modified miRNA precursor in which the miRNA sequence and its complementary sequence are replaced by an amiRNA sequence and its complementary sequence, wherein the amiRNA sequence is (i) fully complementary to a target sequence, (ii) fully complementary to the target sequence except for GU base pairing or (iii) fully complementary to the target sequence in the first ten nucleotides counting from the 5′ end of the amiRNA and wherein the promoter initiates and mediates transcription of the antisense strand of the amiRNA precursor.

2. The nucleic acid construct of claim 1, wherein the promoter is a host cell promoter.

3. The nucleic acid construct of claim 1, wherein a site-specific recombination site is present between the promoter and the polynucleotide.

4. The nucleic acid construct of claim 3, wherein the promoter is a host cell promoter.

5. The nucleic acid construct of claim 1, wherein the miRNA precursor is a plant miRNA precursor.

6. The nucleic acid construct of claim 1, wherein the amiRNA sequence is fully complementary to the target sequence.

7. The nucleic acid construct of claim 1, wherein the amiRNA sequence is fully complementary to the target sequence except for GU base pairing.

8. The nucleic acid construct of claim 1, wherein the target sequence is a RNA of a plant pathogen.

9. The nucleic acid construct of claim 8, wherein the plant pathogen is a plant virus or plant viroid.

10. The nucleic acid construct of claim 9, wherein the target sequence is selected from the group consisting of a sequence of a critical region of a virus, a conserved sequence of a family of viruses and a conserved sequence among members of different viral families.

11. The nucleic acid construct of claim 1, wherein the target sequence is a contiguous sequence in a non-coding region of RNA.

12. The nucleic acid construct of claim 1, wherein the target sequence is a contiguous sequence in a coding region of RNA.

13. The nucleic acid construct of claim 1, wherein the target sequence is a contiguous sequence which overlaps a coding and a non-coding region of RNA.

14. The nucleic acid construct of claim 1, wherein the target sequence comprises a splice site of RNA.

15. A cell comprising the nucleic acid construct of claim 1.

16. The cell of claim 15, wherein the cell is a plant cell.

17. A transgenic plant comprising the nucleic acid of claim 1.

18. A transgenic seed comprising the nucleic acid of claim 1.

19. A cell comprising a host cell promoter operatively linked to a polynucleotide which encodes an artificial miRNA (amiRNA) precursor capable of forming a double-stranded RNA or a hairpin, wherein the amiRNA precursor comprises a modified miRNA precursor in which the miRNA sequence and its complementary (miRNA* strand) sequence are replaced by an amiRNA sequence and its fully complementary sequence, respectively, wherein the amiRNA sequence is (i) fully complementary to a target sequence, (ii) fully complementary to the target sequence except for GU base pairing or (iii) fully complementary to the target sequence in the first ten nucleotides counting from the 5′ end of the amiRNA and wherein the host cell promoter initiates and mediates transcription of the antisense strand of the amiRNA precursor.

20. The cell of claim 19, wherein a site-specific recombination site is present between the host cell promoter and the polynucleotide.

21. The cell of claim 19, wherein the cell is a plant cell.

22. A transgenic plant comprising a host cell promoter operatively linked to a polynucleotide which encodes an artificial miRNA (amiRNA) precursor capable of forming a double-stranded RNA or a hairpin, wherein the amiRNA precursor comprises a modified miRNA precursor in which the miRNA sequence and its complementary (miRNA* strand) sequence are replaced by an amiRNA sequence and its fully complementary sequence, respectively, wherein the amiRNA sequence is (i) fully complementary to a target sequence, (ii) fully complementary to the target sequence except for GU base pairing or (iii) fully complementary to the target sequence in the first ten nucleotides counting from the 5′ end of the amiRNA and wherein the host cell promoter initiates and mediates transcription of the antisense strand of the amiRNA precursor.

23. The transgenic plant of claim 22, wherein a site-specific recombination site is present between the host cell promoter and the polynucleotide.

24. A transgenic seed comprising a host cell promoter operatively linked to a polynucleotide which encodes an artificial miRNA (amiRNA) precursor capable of forming a double-stranded RNA or a hairpin, wherein the amiRNA precursor comprises a modified miRNA precursor in which the miRNA sequence and its complementary (miRNA* strand) sequence are replaced by an amiRNA sequence and its fully complementary sequence, respectively, wherein the amiRNA sequence is (i) fully complementary to a target sequence, (ii) fully complementary to the target sequence except for GU base pairing or (iii) fully complementary to the target sequence in the first ten nucleotides counting from the 5′ end of the amiRNA and wherein the host cell promoter initiates and mediates transcription of the antisense strand of the amiRNA precursor.

25. The transgenic seed of claim 24, wherein a site-specific recombination site is present between the host cell promoter and the polynucleotide.

26. A method for down regulating a target sequence in a cell comprising:
(a) introducing into a cell a polynucleotide which encodes an artificial miRNA (amiRNA) precursor capable of forming a double-stranded RNA or a hairpin, wherein the amiRNA precursor comprises a modified miRNA precursor in which the miRNA sequence and its complementary (miRNA* strand) sequence are replaced by an amiRNA sequence and its fully complementary sequence, respectively, wherein the amiRNA sequence is (i) fully complementary to a target sequence, (ii) fully complementary to the target sequence except for GU base pairing or (iii) fully complementary to the target sequence in the first ten nucleotides counting from the 5′ end of the amiRNA, wherein the polynucleotide is operably linked to a promoter, and wherein the promoter initiates and mediates transcription of the antisense strand of the amiRNA precursor and
(b) growing the cell of (a) under conditions wherein the promoter initiates and mediates transcription of the antisense strand of the amiRNA precursor, the amiRNA is produced and the RNA containing the target sequence is cleaved.

27. The method of claim 26, wherein the promoter is a host cell promoter.

28. The method of claim 26, wherein a site-specific recombination site is present between the promoter and the polynucleotide.

29. The method of claim 28, wherein the promoter is a host cell promoter.

30. The method of claim 26, wherein the cell is a plant cell.

31. The method of claim 26, wherein the RNA containing the target sequence is cleaved within the target sequence.

32. The method of claim 31, wherein the modified miRNA is a plant miRNA modified to be fully complementary to the target sequence.

33. The method of claim 31, wherein the modified miRNA is a plant miRNA modified to be fully complementary to the target sequence except for the use of GU base pairing.

34. The method of claim 26, wherein the target sequence is a contiguous sequence in an RNA of a plant pathogen.

35. The method of claim 34, wherein the plant pathogen comprising the target sequence is a plant virus or plant viroid.

36. The method of claim 35, wherein the target sequence is selected from the group consisting of a sequence of a critical region of a virus, a conserved sequence of a family of viruses and a conserved sequence among members of different viral families.

37. The method of claim 26, wherein the target sequence is a contiguous sequence in a coding region of RNA.

38. The method of claim 26, wherein the target sequence is a contiguous sequence in a non-coding region of RNA.

39. The method of claim 26, wherein the target sequence is a contiguous sequence which overlaps a coding region and a non-coding region of RNA.

40. The method of claim 26, wherein the target sequence comprises a splice site of RNA.

41. The method of claim 26, wherein the nucleic acid construct is inserted into an intron of a gene or transgene of the cell.

42. A method for producing artificial miRNA (amiRNA) in a cell comprising:
(a) introducing into a cell a polynucleotide which encodes an artificial miRNA (amiRNA) precursor capable of forming a double-stranded RNA or a hairpin, wherein the amiRNA precursor comprises a modified miRNA precursor in which the miRNA sequence and its complementary (miRNA* strand) sequence are replaced by an amiRNA sequence and its fully complementary sequence, respectively, wherein the amiRNA sequence is (i) fully complementary to a target sequence, (ii) fully complementary to the target sequence except for GU base pairing or (iii) fully complementary to the target sequence in the first ten nucleotides counting from the 5′ end of the amiRNA, wherein the polynucleotide is operably linked to a promoter, and wherein the promoter initiates and mediates transcription of the antisense strand of the amiRNA precursor and
(b) growing the cell of (a) under conditions wherein the promoter initiates and mediates transcription of the antisense strand of the amiRNA precursor and the amiRNA is produced.

43. The method of claim 42, wherein the promoter is a host cell promoter.

44. The method of claim 42, wherein a site-specific recombination site is present between the promoter and the polynucleotide.

45. The method of claim 44, wherein the promoter is a host cell promoter.

46. The method of claim 42, wherein the cell is a plant cell.

47. A method to assay for production of an artificial miRNA comprising the steps of:
(a) identifying a precursor miRNA containing a miRNA sequence and a complementary miRNA sequence (a miRNA* strand sequence),
(b) introducing into a cell a nucleic acid construct comprising a promoter operably linked to a polynucleotide encoding an artificial miRNA (amiRNA) precursor wherein the amiRNA precursor comprises the following:
(1) an artificial miRNA sequence replacing the endogenous miRNA sequence;
(2) a fully complementary sequence of the artificial miRNA replacing the endogenous miRNA* strand sequence; and
wherein the promoter initiates and mediates transcription of the antisense strand of the amiRNA precursor;
(c) growing the cell of (b) under conditions wherein the promoter initiates and mediates transcription of the antisense strand of the amiRNA precursor; and
(d) assaying for production of the artificial miRNA.

48. The method of claim 47, wherein the amiRNA precursor of step (b) further comprises:
(3) replacement of one or more potential GU base pairs in the predicted secondary structure of the amiRNA precursor with one or more CG or AT base pairs.

49. The method of claim 47, wherein the amiRNA precursor of step (b) further comprises:
(3) replacement of one or more potential GC base pairs in the predicted secondary structure of the amiRNA precursor with one or more AT base pairs.

50. The method of claim 47, wherein the amiRNA precursor of step (b) further comprises:
(3) replacement of one or more potential AT base pairs in the predicted secondary structure of the amiRNA precursor with one or more CG base pairs.

51. A method for down regulating an RNA containing a target sequence in a plant comprising:
(a) introducing into a plant cell a polynucleotide which encodes an artificial miRNA (amiRNA) precursor capable of forming a double-stranded RNA or a hairpin, wherein the amiRNA precursor comprises a modified miRNA precursor in which the miRNA sequence and its complementary (miRNA* strand) sequence are replaced by an amiRNA sequence and its fully complementary sequence, respectively, wherein the amiRNA sequence is (i) fully complementary to a target sequence, (ii) fully complementary to the target sequence except for GU base pairing or (iii) fully complementary to the target sequence in the first ten nucleotides counting from the 5′ end of the amiRNA, wherein the polynucleotide is operably linked to a promoter, and wherein the promoter initiates and mediates transcription of the antisense strand of the amiRNA precursor,
(b) regenerating a plant from the plant cell of (a) wherein the plant comprises in its genome the nucleic acid construct,
(c) growing the plant of (b) under conditions wherein the promoter initiates and mediates transcription of the antisense strand of the amiRNA precursor, the amiRNA is produced and the RNA containing the target sequence is cleaved.

52. The method of claim 51, wherein the promoter is a host cell promoter.

53. Use of a synthetic pre-miRNA to generate one or more mature miRNAs in transformants to down regulate one or more target genes.

54. The use of claim 53, wherein the synthetic pre-miRNA is selected from the group consisting of a modified native pre-miRNA, an antisense native pre-miRNA, an antisense native modified pre-miRNA and an artificial designed pre-miRNA.

55. The use of claim 53, wherein the mature miRNA is a native mature miRNA or an amiRNA.

56. The use of claim 53, wherein the transformant is a transformed plant cell, a transformed non-human animal cell, a transformed human cell in vitro, a transformed animal stem cell or a transformed human stem cell.

57. The use of claim 53, wherein the transformant is a transformed plant or a transformed non-human animal.
